(19) United States
(12) Patent Application Publication
(10) Pub. No.: US 2015/0283060 A1
(43) Pub. Date: Oct. 8, 2015

(54) FOAM AEROSOL FOR INCREASING VOLUME

(71) Applicant: Henkel AG & Co. KGaA, Duesseldorf (DE)

(72) Inventors: Diane Metten, Hamburg (DE); Bernd Richters, Hamburg (DE); Rene Scheffler, Ellerau (DE)

(21) Appl. No.: 14/742,805

(22) Filed: Jun. 18, 2015

Related U.S. Application Data
(63) Continuation of application No. PCT/EP2013/073679, filed on Nov. 13, 2013.

(30) Foreign Application Priority Data
Dec. 20, 2012 (DE) 10 2012 223 971.5

(51) Int. Cl. A61K 8/894; A61K 8/81; A61Q 5/06

(52) U.S. Cl. CPC A61K 8/894; A61Q 5/06; A61K 8/8152; A61K 2800/20; A61K 2800/594

(57) ABSTRACT

Foams produced from a cosmetic agent, containing, in a cosmetically acceptable carrier
a) a polymer combination of at least one copolymer A, comprising
   (a1) at least one repetitive structural unit having at least one a silicone-containing side chain,
   (a2) at least one structural unit of formula (a2)

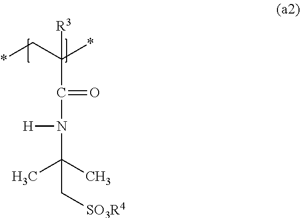

where R³ represents a hydrogen atom or a methyl group and R⁴ represents a hydrogen atom or an equivalent of a cation, and
(a3) at least one structural unit of formula (a3)

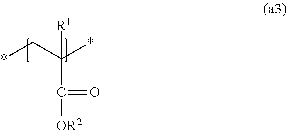

where R¹ represents a hydrogen atom or a methyl group and
R² represents a hydrogen atom, an equivalent of a cation, or a (C₁-C₄) alkyl group, and
at least one copolymer B constituted of the monomers
   B1) n-butyl methacrylate
   B2) methacrylic acid
   B3) ethyl acrylate
   B4) ethyl methacrylate
   and optionally additional monomers, and
b) at least one propellant, and
c) water,
   are especially suitable for increasing the volume of keratinous fibers.

### FIELD OF THE INVENTION

The present invention generally relates to the reshaping and shape fixation of keratinous fibers, particularly human hair. Keratinous fibers are understood in principle to be all animal hair, for example, wool, horsehair, angora hair, fur, feathers, and products or textiles manufactured therefrom. Preferably, according to the invention, however, the keratinous fibers are human hair.

### BACKGROUND OF THE INVENTION

The use of polymers in a wide variety cosmetic agents is very common. They are found in agents for the treatment of the skin, as well as in agents for the treatment of hair, in agents that are washed off or out immediately after use, so-called rinse-off products, as well as in agents remaining on the skin or hair, so-called leave-on products. In this regard, the polymers are used for a wide variety of reasons and in each case specific properties of the polymers are utilized. In agents for skin treatment, in shampoos, hair conditioners, and hair treatments, the thickening or nourishing properties of the polymers are often in the forefront. In agents for the temporary deformation of keratinic fibers, also called styling agents below, primarily the film-forming and/or strengthening effects are sought apart from these properties. Polymers are also often used as additives to improve or in the first place to make possible the deposition and fixation of other active substances and components on the skin or hair. Thus, for example, the rubbing fastness and durability of the coloring can be increased by addition of suitable polymers to hair dyes.

It is necessary in the case of styling agents that the employed polymers give the treated hair a strongest possible hold. However, in addition to a high degree of hold, styling agents can desirably satisfy a wide range of further requirements. These can be divided roughly into properties on the hair, properties of the particular formulation, e.g., properties of the foam, gel, or sprayed aerosol, and properties related to the handling of the styling agent, particular importance being attached to the properties on the hair. Mention can be made in particular of humidity resistance, low tackiness, and a balanced conditioning effect. Furthermore, as far as possible a styling agent should be universally suitable for all hair types.

The temporary deformation of keratinous fibers usually seeks to fix a shape newly impressed on the fiber population, in the case of hair a new hairstyle. This hairstyle need not necessarily mean a revolutionary inasmuch as a drastic change in shape. Subtle effects such as, e.g., volume effects are often aspired to, which impart more fullness through more volume particularly on a fiber population of thin keratinic fibers. As a result, the fibers no longer hang down flat and close to the scalp, but take on more volume and impart a greater fullness. If the keratinic fibers are hair, thus hair fullness and hair volume can be achieved in addition in that the hair in the hairline area does not immediately fall closely to the scalp, but first projects radially from the scalp due to the hair fixation by means of the fixing polymer, before the hair tip region of the fiber falls downward due to gravity.

The international patent application WO 2011/057882 A1 describes a tetrapolymer with good cosmetic properties, particularly in the field of hair care and hair styling. Said tetrapolymer is obtained by polymerizing the monomers n-butyl methacrylate, methacrylic acid, ethyl acrylate, and ethyl methacrylate. WO 2011/057882 A1 describes furthermore the combination of said special tetrapolymer with other film-forming polymers in agents for the shaping of keratinic fibers. The group of these other film-forming polymers includes, inter alia, also a copolymer of N-vinylpyrrolidone and vinyl acetate, called Luviskol® VA 37. Suitable polymer mixtures during use in agents for the temporary shaping of keratinic fibers proved to be in need of improvement, however.

The object of the present invention was to provide a cosmetic agent that imparts more volume and fullness on keratinous fibers, especially human hair. The volumizing should last for a long time without having to forego flexibility and good humidity resistance, particularly resistance to sweat and water.

It was possible to achieve said technical object by the cosmetic agent of the first subject of the invention.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Cosmetic agent, containing in a cosmetically acceptable carrier: a) a polymer combination of at least one copolymer A, comprising (a1) at least one repeating structural unit with at least one a silicone-containing side chain, (a2) at least one structural unit of formula (a2)

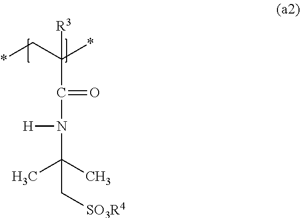

where R³ stands for a hydrogen atom or a methyl group and R⁴ stands for a hydrogen atom or an equivalent of a cation, (a3) at least one structural unit of formula (a3)

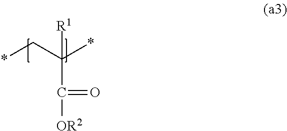

where R¹ stands for a hydrogen atom or a methyl group and R² stands for a hydrogen atom, an equivalent of a cation, or a (C₁-C₄) alkyl group, and at least one copolymer B of the monomers B1) n-butyl methacrylate, B2) methacrylic acid, and B3) ethyl acrylate, and optionally additional monomers; and b) at least one propellant; and c) water.

### BRIEF DESCRIPTION OF THE DRAWINGS

The present invention will hereinafter be described in conjunction with the following drawing FIGURES, wherein like numerals denote like elements, and

FIG. 1 is a generalized diagram showing the setup for a hairline volume measurement.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the invention is a cosmetic agent, containing in a cosmetically acceptable carrier

a) a polymer combination of at least one copolymer A, comprising
(A1) at least one repeating structural unit with at least one silicone-containing side chain,
(A2) at least one structural unit of formula (a2)

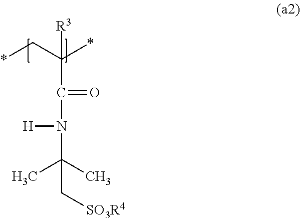

where R³ stands for a hydrogen atom or a methyl group and R⁴ stands for a hydrogen atom or an equivalent of a cation,
(A3) at least one structural unit of formula (a3)

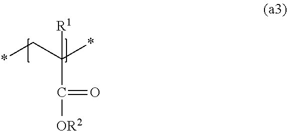

where R¹ stands for a hydrogen atom or a methyl group and
R² stands for a hydrogen atom, an equivalent of a cation, or a (C₁-C₄) alkyl group, and at least one copolymer B of the monomers
   B1) n-butyl methacrylate
   B2) methacrylic acid
   B3) ethyl acrylate
   and optionally additional monomers, and
      b) at least one propellant, and
      c) water.

In particular, a foam is especially preferred according to the invention, which is formed from the agent according to the invention of the first subject of the invention or all preferred embodiments described below.

Suitable salts of the aforementioned structural unit (a2) are, for example, alkali metal salts (such as, for example, sodium, potassium, and lithium salts), alkaline earth metal salts (such as, for example, calcium and magnesium salts), ammonium salts (NH₄⁺), and salts obtained by reaction with alkanolamines (such as, for example, with 2-amino-2-methyl-1-propanol, 2-aminoethanol, diethanolamine, triethanolamine, triethylamine). When “salts” are discussed below in relation to copolymer A, these aforementioned salts are preferred (if not explicitly stated otherwise). Aforementioned cations are cations within the meaning of formulas (a2) and (a3) and are used accordingly in the equivalent with the preservation of electroneutrality.

It turned out as especially preferably according to the invention, if the silicone-containing side chains of the anionic polymer are selected from representatives of the group of silicone copolyols. Silicone copolyols are silicones containing in their structure at least one unit if one or more alkylene glycols, especially ethylene glycol and/or propylene glycol.

Said copolymer A is preferably selected from at least one polymer obtained from monomers (i), (ii), and (iii)
(i) at least one monomer, carrying an olefinically unsaturated group and a silicone moiety, and
(ii) at least one monomer of formula (a2-m),

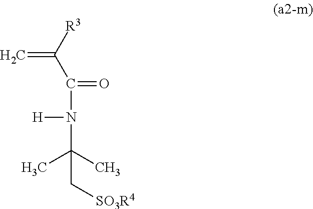

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt.

In all aforementioned embodiments, to preserve the structural unit (a1) preferably at least one monomer selected from the group, formed by compounds of formulas (A1) and (A2), is used as monomer (i)

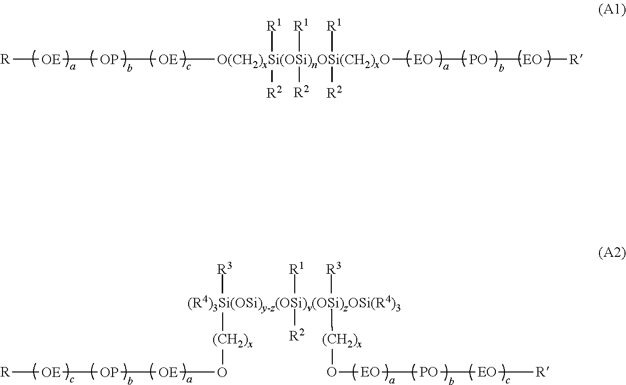

where
R¹ and R² independently of one another stand for a (C₁ to C₃₀) alkyl group, a (C₁ to C₂₀) alkyl group substituted with at least one halogen atom (e.g., —CCl₃, —CBr₃, —CF₃), a (C₃ to C₈) cycloalkyl group, a (C₆ to C₁₄) aryl group, or (C₂ to C₂₀) alkenyl group,
R³ stands for a (C₁ to C₃₀) alkyl group, a (C₁ to C₂₀) alkyl group substituted with at least one halogen atom (e.g., —CCl₃, —CBr₃, —CF₃), a (C₃ to C₈) cycloalkyl group, a (C₆ to C₁₄) aryl group, or (C₂ to C₂₀) alkenyl group,
R⁴ independently of one another stands for a (C₁ to C₃₀) alkyl group, a (C₆ to C₁₄) aryl group, or a (C₂ to C₂₀) alkenyl group,
R and R′ at least one of the two groups R or R′ stands for an ester group of an olefinically unsaturated carboxylic acid according to one of formulas (Est1) to (Est7), whereby the other group also denotes one of these ester groups of formulas (Est1) to (Est7) or stands for a hydrogen atom,

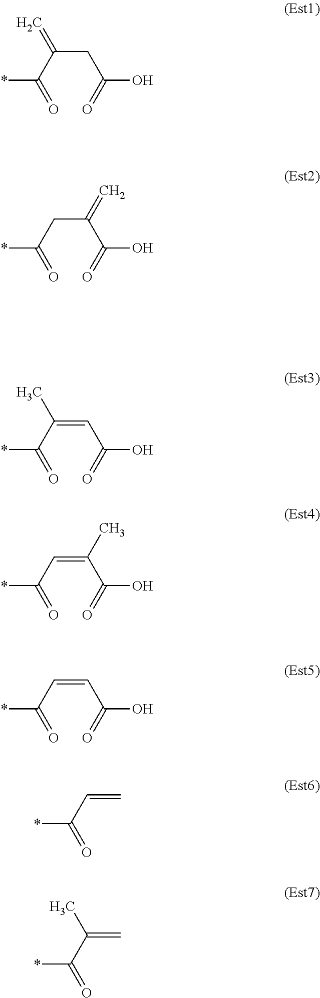

E stands for an ethane-1,2-diyl group,
P stands for a propane-1,2-diyl group or a propane-1,3-diyl group,
a, b and c independently of one another stand for a number from 0 to 100,
n stands for a number from 0 to 1000,
x stands for a number 2 or 3,
v stands for a number from 0 to 200,
y stands for a number from 1 to 200, and
z is less than or equal to y.

The EO or PO or OE or OP fragments can be present xx or be randomly distributed in the monomer.

Within the scope of a further preferred embodiment, the agent according to the invention contains as copolymer A at least one polymer formed from at least the monomers (i) to (iii),
(i) for the preservation of the structural unit (a1) at least one monomer with a silicone side chain selected from the group formed from compounds of formulas (A1) and (A2)

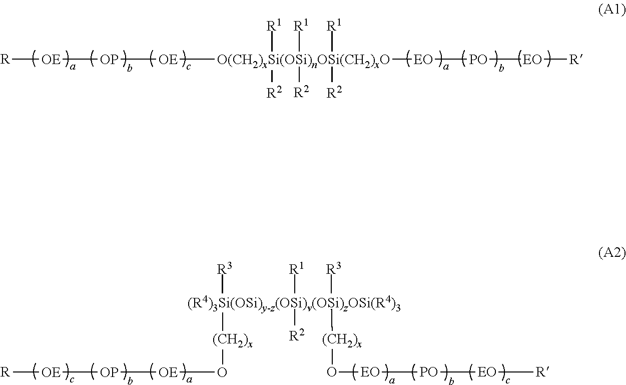

where
R¹ and R² independently of one another stand for a (C₁ to C₃₀) alkyl group, a (C₁ to C₂₀) alkyl group substituted with at least one halogen atom (e.g., —CCl₃, —CBr₃, —CF₃), a (C₃ to C₈) cycloalkyl group, a (C₆ to C₁₄) aryl group, or (C₂ to C₂₀) alkenyl group,
R³ stands for a (C₁ to C₃₀) alkyl group, a (C₁ to C₂₀) alkyl group substituted with at least one halogen atom (e.g., —CCl₃, —CBr₃, —CF₃), a (C₃ to C₈) cycloalkyl group, a (C₆ to C₁₄) aryl group, or (C₂ to C₂₀) alkenyl group,
R⁴ independently of one another stands for a (C₁ to C₃₀) alkyl group, a (C₆ to C₁₄) aryl group, or a (C₂ to C₂₀) alkenyl group,
R and R′ at least one of the two groups R or R′ stands for an ester group of an olefinically unsaturated carboxylic acid according to one of formulas (Est1) to (Est7), whereby the other group also denotes one of these ester groups of formulas (Est1) to (Est7) or stands for a hydrogen atom,

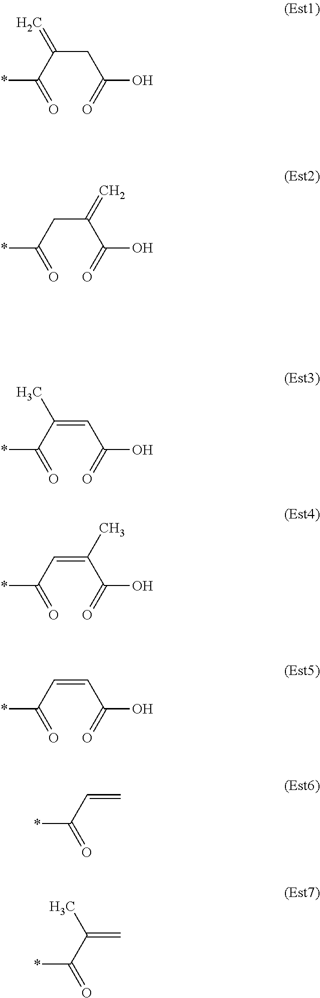

E stands for an ethane-1,2-diyl group,
P stands for a propane-1,2-diyl group or a propane-1,3-diyl group,
a, b and c independently of one another stand for a number from 0 to 100,
n stands for a number from 0 to 1000,
x stands for a number 2 or 3,
v stands for a number from 0 to 200,
y stands for a number from 1 to 200, and
z is less than or equal to y,
(ii) at least one monomer according to formula (a2-m)

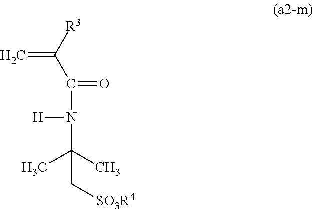

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt.

It is especially preferable according to the invention if x according to formula (A1) or (A2) stands for the number 3.

Within the meaning of all aforementioned embodiments, it is very especially preferable, if the monomer for the preservation of structural unit (a1) is selected from at least one monomer from the group, formed from formulas (A1-1), (A1-2), (A2-1), and (A2-2)

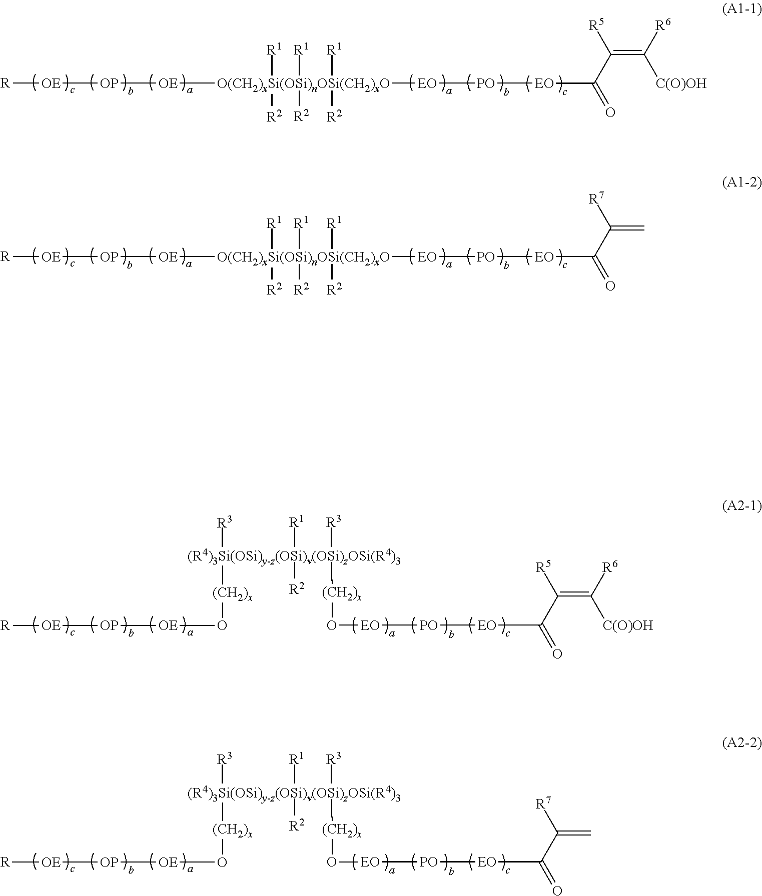

where
R¹, R², R³, R⁴, E, P, a, b, c, x, n, v, y, and z are defined according to formulas (A1) and (A2),
R⁷ stands for a hydrogen atom or a methyl group,
where
one of the groups R⁵ or R⁶ stands for a methyl group and the other for a hydrogen atom and R stands for a hydrogen atom or for a group of formulas (Esta) or (Est9),

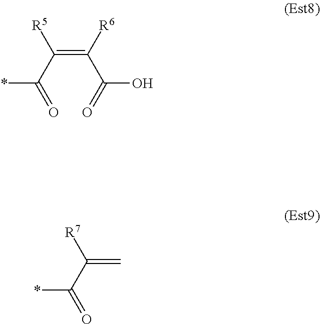

in which R⁵, R⁶, and R⁷ are defined as above.

According to formulas (A1) or (A2) or (A1-1) or (A1-2) or (A2-1) or (A2-2), preferably suitable monomers are those in which x stands for the number 3.

A very especially preferred anionic polymer with silicone-containing side chains is Polyacrylate-32. It is sold, for example, under the trade name Fixate® Design (32% by weight of active polymeric substance, INCI name: Polyacrylate-32) by Lubrizol.

Preferred agents according to the invention are characterized in that the proportion by weight of copolymer A in the total weight of the cosmetic agent is between 0.5 and 10% by weight, preferably between 3.0 and 8.0% by weight.

Copolymers B employed according to the invention are obtained by polymerizing the monomers: n-butyl methacrylate, methacrylic acid, and ethyl acrylate, and optionally other monomers. Preferred copolymers B consist of at least 90% by weight, preferably at least 95% by weight, and particularly at least 97% by weight of the monomers: n-butyl methacrylate, methacrylic acid, and ethyl acrylate. Especially preferred are copolymers B that were obtained as terpolymers exclusively from the monomers: n-butyl methacrylate, methacrylic acid, and ethyl acrylate.

In a preferred embodiment, copolymers B based on their weight have 80 to 80% by weight of n-butyl methacrylate, 10 to 30% by weight of methacrylic acid, and 5 to 15% by weight of ethyl acrylate, whereby copolymers B with a proportion by weight of the monomers of 65 to 75% by weight of n-butyl methacrylate, 15 to 25% by weight of methacrylic acid, and 8 to 12% by weight of ethyl acrylate are especially preferred. Very especially preferred copolymers B have a proportion by weight of the monomers of 67 to 72% by weight of n-butyl methacrylate, 18 to 23% by weight of methacrylic acid, and 9 to 11% by weight of ethyl acrylate.

Copolymers B preferably have a molecular weight of about 100 kDa. The glass transition temperature is preferably between 80 and 120° C., particularly between 85 to 105° C.

The above-described copolymers B are sold, for example, under the name Tilamar® Fix A140 (INCI: Acrylates copolymer; CAS number: 26715-43-5) by the company DSM.

Preferred agents according to the invention are characterized in that, based on the total weight of the agent according to the invention, copolymer B is contained in the amount of 0.5 to 10% by weight, preferably of 3.0 to 8.0% by weight.

An especially preferred agent according to the invention is characterized in that, based on its total weight, it contains the polymer combination of copolymer A and copolymer B in a total amount of 1.0 to 11.0% by weight, preferably 3.0 to 8.0% by weight.

Copolymer A and copolymer B are used in the cosmetic agent according to the invention preferably in a partially neutralized or neutralized form. Preferably at least one alkanolamine is used for neutralization. The alkanolamines that can be used as alkalizing agents are preferably selected from primary amines having a C₂-C₆ alkyl parent substance, which carries at least one hydroxyl group. Especially preferred alkanolamines are selected from the group, formed by 2-aminoethan-1-ol (monoethanolamine), tris(2-hydroxyethyl)amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, and 2-amino-2-methylpropane-1,3-diol. Alkanolamines very especially preferred according to the invention are selected from the group of 2-aminoethan-1-ol, 2-amino-2-methylpropan-1-ol, and 2-amino-2-methylpropane-1,3-diol. 2-Amino-2-methylpropanol has proven to be an especially suitable neutralizing agent. Cosmetic agents preferred according to the invention therefore contain 2-amino-2-methylpropanol. 2-Amino-2-methylpropanol is employed in the agent according to the invention preferably in an amount that does not exceed the amount necessary for the neutralization of copolymer A. Preferably, the amounts, used in the agents according to the invention, of 2-amino-2-methylpropanol are 80 to 100%, especially preferably 90 to 100%, and especially 95 to 100% of the amount required for the complete neutralization of copolymer A. In a preferred embodiment, the proportion by weight of 2-amino-2-methylpropanol in the total weight of the cosmetic agent is 0.05 to 4.0% by weight, preferably 0.1 to 2.5% by weight, and especially 0.2 to 2.0% by weight.

The cosmetic agents according to the invention can contain, in addition to the above-described copolymers A and B, at least one further fixing polymer, which is different from the above-described copolymers A and B. Fixing polymers contribute to holding the impressed shape of the fiber population, e.g., the overall hairstyle. These polymers are simultaneously also film-forming polymers and therefore generally typical substances for styling hair treatment products such as hair setting products, hair mousses, hair waxes, and hair sprays. The film formation in this case can be quite selective and only connect a few fibers with one another.

Film-forming polymers are understood to be such polymers that upon drying leave behind a continuous film on the skin, hair, or nails. Such film formers can be used in very different cosmetic products such as, for example, facial masks, makeup, hair setting products, hair sprays, hair gels, hair waxes, hair treatments, shampoos, or nail polish. Such polymers are preferred in particular that have a sufficient solubility in water or water/alcohol mixtures, in order to be present in the agent according to the invention in completely dissolved form. The film-forming polymers can be synthetic or natural in origin.

According to the invention, film-forming polymers are understood further to be such polymers that during use in 0.01 to 20% by weight aqueous, alcoholic, or aqueous/alcoholic solution are capable of depositing a transparent polymer film on hair

The additional fixing polymers are preferably selected from nonionic fixing polymers, amphoteric fixing polymers, cationic fixing polymers, and anionic fixing polymers, especially preferably from nonionic fixing polymers, anionic fixing polymers, and amphoteric fixing polymers.

The agent according to the invention contains preferably in addition at least one fixing nonionic polymer, containing at least one structural unit, selected from the group of structural units of formulas (M1) to (M6)

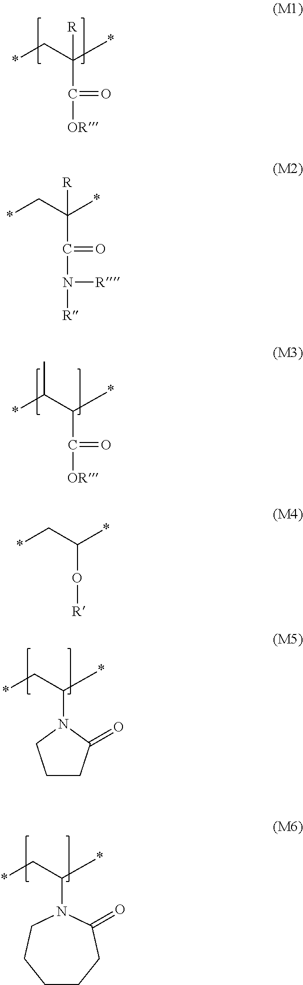

where
R stands for a hydrogen atom or a methyl group,
R′ stands for a hydrogen atom or a (C₁ to C₄) acyl group,
R″ and R″″ independently of one another stand for a (C₁ to C₇) alkyl group or a hydrogen atom
R′″ stands for a linear or branched (C₁ to C₄) alkyl group or a (C₂ to C₄) hydroxyalkyl group.

According to the above formulas and all following formulas, a chemical bond labeled with the symbol * stands for a free valence of the corresponding structural fragment.

The additional fixing nonionic polymers (b) are contained in the agent according to the invention preferably in an amount of 0.1% by weight to 20.0% by weight, more preferably 0.2% by weight to 15.0% by weight, and even more preferably 0.5% by weight to 10.0% by weight, in each case based on the weight of the agent according to the invention.

Preferred nonionic film-forming and/or nonionic hair-fixing polymers (b) are homo- or copolymers made up of at least one of the following monomers: N-vinylpyrrolidone, N-vinylcaprolactam, vinyl esters (such as, e.g., vinyl acetate, vinyl alcohol), acrylamide, methacrylamide, alkyl and dialkyl acrylamide (particularly N-methyl- and N,N-dimethylacrylamide), alkyl and dialkyl methacrylamide (particularly N-methyl- and N,N-dimethylmethacrylamide), alkyl acrylate, and alkyl methacrylate, whereby in each case the alkyl groups of these monomers are selected from (C₁ to C₃) alkyl groups.

Nonionic polymers especially suitable for the agents according to the invention, based on ethylenically unsaturated monomers, contain at least one of the following structural units

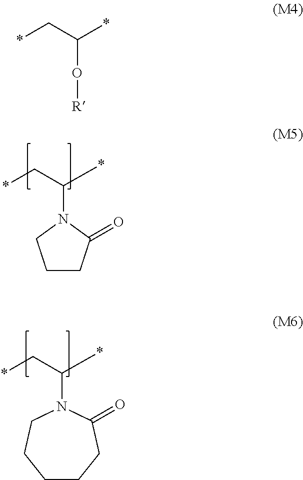

where R¹ stands for a hydrogen atom or a (C₁ to C₃₀) acyl group, particularly for a hydrogen atom or an acetyl group.

Suitable in particular are homopolymers of vinylcaprolactam or of vinylpyrrolidone (such as, for example, Luviskol® K 90 or Luviskol® K 85 from the company BASF SE), copolymers of vinylpyrrolidone and vinyl acetate (in this case, it is again preferable, if the molar ratio of the structural units comprising the monomer N-vinylpyrrolidone to the structural units comprising the monomer vinyl acetate of the polymer is in the range of 20 to 80 to 80 to 20, particularly of 30 to 70 to 60 to 40; for example, sold under the trademark Luviskol® VA 37, Luviskol® VA 55, Luviskol® VA 64, and Luviskol® VA 73 from BASF SE), terpolymers of vinylpyrrolidone, vinyl acetate, and vinyl propionate, polyacrylamides (such as, for example, Akypomine® P 191 from CHEM-Y), polyvinyl alcohols (which are sold, for example, under the trade names Elvanol® by Du Pont or Vinol® 523/540 by Air Products), terpolymers of vinylpyrrolidone, methacrylamide, and vinylimidazole (such as, for example, Luviset® Clear from BASF SE).

Agents, which contain as film-forming nonionic and/or fixing nonionic polymer (b) at least one polymer selected from the group, formed from
polyvinylpyrrolidone,
copolymers of N-vinylpyrrolidone and vinyl esters of carboxylic acids having 2 to 18 carbon atoms, especially of N-vinylpyrrolidone and vinyl acetate,
   are very especially preferred according to the invention.

Within the scope of a possible embodiment, the agent according to the invention contains as an additional fixing polymer at least one fixing cationic polymer.

Cationic polymers are understood to be polymers with a group that can be cationic “temporarily” or “permanently” in the main and/or side chain. Polymers that independent of the pH of the agent have a cationic group are designated as “permanently cationic” according to the invention. These are usually polymers that contain a quaternary nitrogen atom, for example, in the form of an ammonium group. Preferred cationic groups are quaternary ammonium groups. In particular polymers in which the quaternary ammonium group is bound via a C₁₋₄ hydrocarbon group to a polymer main chain made up of acrylic acid, methacrylic acid, or derivatives have proven to be especially suitable.

A cationic fixing polymer preferably suitable according to the invention is at least one cationic fixing polymer, which comprises at least one structural unit of formula (M5) and at least one structural unit of formula (V) and optionally at least one structural unit of formula (VI)

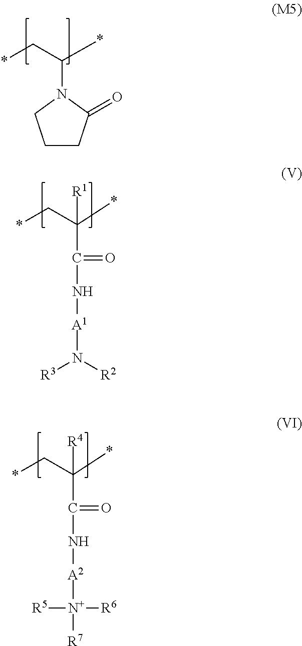

where
R¹ and R⁴ independently of one another stand for a hydrogen atom or a methyl group,
A¹ and A² independently of one another stand for a group ethanol-1,2-diyl, propane-1,3-diyl, or butane-1,4-diyl,
R², R³, R⁵ and R⁶ independently of one another stand for a (C₁ to C₄) alkyl group,
R⁷ stands for a (C₈ to C₃₀) alkyl group.

All possible physiologically acceptable anions, such as, for example, chloride, bromide, hydrogen sulfate, methyl sulfate, ethyl sulfate, tetrafluoroborate, phosphate, hydrogen phosphate, dihydrogen phosphate, or p-toluene sulfonate, and triflate, are used to compensate the positive charge of monomer (VI).

Such compounds are available, for example, as
copolymers of N-vinylpyrrolidone, N-(3-dimethylaminopropyl)methacrylamide and 3-(methacryloylamino)propyllauryl dimethylammonium chloride (INCI name: Polyquaternium-55), which is sold by ISP, for example, under the trade names Styleze W 10 or Styleze W 20 (10 or 20% by weight of active substance in an ethanol/water mixture).
copolymers of N-vinylpyrrolidone, N-vinylcaprolactam, N-(3-dimethylaminopropyl)-methacrylamide, and 3-(methacryloylamino)propyllauryl dimethylammonium chloride (INCI name: Polyquaternium-69), which is sold by ISP, for example, under the trade name AquaStyle® 300 (28-32% by weight of active substance in an ethanol/water mixture).

Further cationic polymers, used preferably in the agents according to the invention, are the so-called “temporarily cationic” polymers. These polymers typically contain an amino group, which is present at certain pH values as a quaternary ammonium group and thus cationically.

Temporarily cationic polymers preferably suitable within the meaning of the invention are also those that have at least one structural unit of formulas (M1-1) to (M1-8)

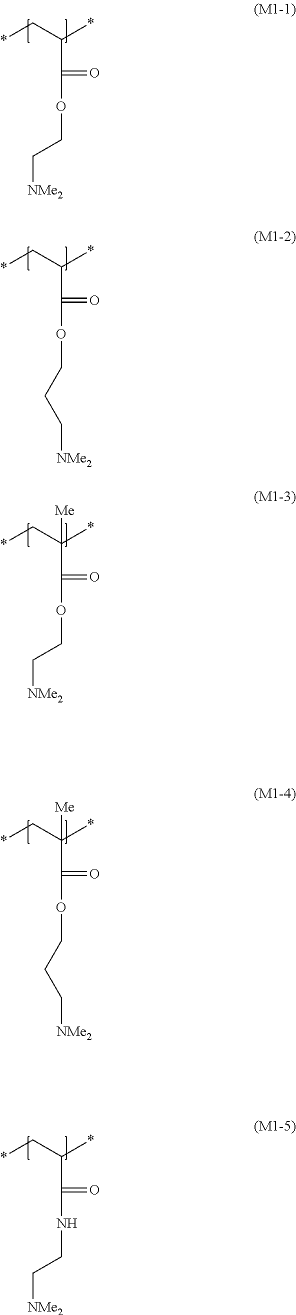

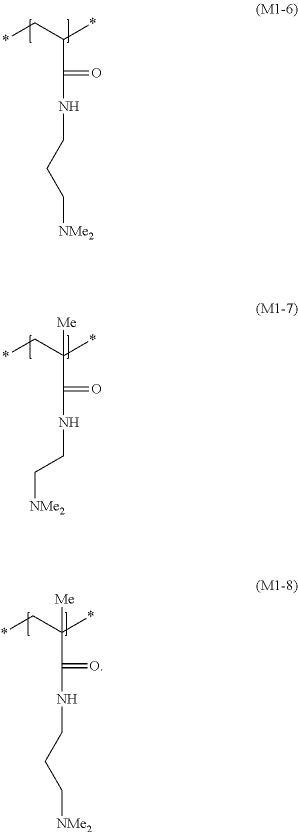

In this regard, such copolymers are again preferred that contain at least one structural unit of formulas (M1-1) to (M1-8) and in addition at least one structural unit of formula (M10),

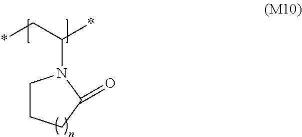

where
n denotes 1 or 3.

In this case, the group of polymers
vinylcaprolactam/vinylpyrrolidone/dimethylaminoethyl methacrylate copolymer (for example, INCI name: Vinyl Caprolactam/PVP/Dimethylaminoethyl Methacrylate Copolymer under the trade name Gaffix® VC 713 (ISP)),
vinylpyrrolidone/vinylcaprolactam/dimethylaminopropyl methacrylamide copolymer (for example, INCI name: VP/Vinyl Caprolactam/DMAPA Acrylates Copolymer under the trade name Aquaflex® SF-40 (ISP)),
vinylcaprolactam/vinylpyrrolidone/dimethylaminoethyl methacrylate copolymer (for example, as 35-39% solid in ethanol in the Ruin of the commercial product Advantage LC E with the INCI name: Vinyl Caprolactam/VP/Dimethylaminoethyl Methacrylate Copolymer, Alcohol, Lauryl Pyrrolidone (ISP)),
vinylpyrrolidone/dimethylaminopropyl methacrylamide copolymer (for example, INCI name: VP/DMAPA Acrylates Copolymer under the trade name Styleze CC-10 (10% by weight of active substance) (ISP)),
   is a preferred list for the selection of at least one or more polymers therefrom.

To intensify the effect of the invention, the agents according to the invention preferably contain in addition at least one surfactant, whereby nonionic, anionic, cationic, and ampholytic surfactants are suitable in principle. It is preferred according to the invention, if the agent according to the invention contains in addition at least one surfactant, selected from at least one compound from of the group, formed from anionic surfactants, nonionic surfactants, and amphoteric surfactants. The group of the ampholytic or also amphoteric surfactants comprises zwitterionic surfactants and ampholytes. According to the invention, the surfactants can already have an emulsifying effect.

The additional surfactants are contained in the agent according to the invention preferably in an amount of 0.01% by weight to 5% by weight, especially preferably 0.05% by weight to 0.5% by weight, in each case based on the weight of the agent.

It has proven as especially preferable, if the agents according to the invention contain in addition at least one nonionic surfactant.

Nonionic surfactants contain as the hydrophilic group, e.g., a polyol group, a polyalkylene glycol ether group, or a combination of polyol and polyglycol ether groups. Such compounds are, for example,
addition products of 2 to 100 mol of ethylene oxide and/or 1 to 5 mol of propylene oxide to linear and branched fatty alcohols having 8 to 30 C atoms, to fatty acids having 8 to 30 C atoms, and to alkylphenols having 8 to 15 C atoms in the alkyl group,
addition products, end-capped with a methyl or C₂-C₆ alkyl group, of 2 to 50 mol of ethylene oxide and/or 1 to 5 mol of propylene oxide to linear and branched fatty alcohols having 8 to 30 C atoms, to fatty acids having 8 to 30 C atoms, and to alkylphenols having 8 to 15 C atoms in the alkyl group, such as, for example, the types obtainable under the marketing names Dehydol® LS and Dehydol® LT (Cognis),
C₁₂-C₃₀ fatty acid monoesters and diesters of addition products of 1 to 30 mol of ethylene oxide to glycerol,
addition products of 5 to 60 mol of ethylene oxide to castor oil and hydrogenated castor oil,
polyol fatty acid esters, such as, for example, the commercial product Hydagen HSP (Cognis) or Sovermol types (Cognis),
alkoxylated triglycerides,
alkoxylated fatty acid alkyl esters of formula (E4-I)

R¹CO—(OCH₂CHR²)_{w}OR³ (E4-I)
in which R¹CO stands for a linear or branched, saturated and/or unsaturated acyl group having 6 to 22 carbon atoms, R² stands for hydrogen or methyl, R³ stands for linear or branched alkyl groups having 1 to 4 carbon atoms, and w stands for a number from 1 to 20,
amine oxides, hydroxy mixed ethers, as they are described, for example, in DE-OS 19738866,
sorbitan fatty acid esters and addition products of ethylene oxide to sorbitan fatty acid esters, such as, for example, polysorbates,
sugar fatty acid esters and addition products of ethylene oxide to sugar fatty acid esters,
addition products of ethylene oxide to fatty acid alkanolamides and fatty amines,
sugar surfactants of the type alkyl and alkenyl oligoglycosides according to formula (E4-II),

R⁴O-[G]ₚ (E4-II)
in which R⁴ stands for an alkyl or alkenyl group having 4 to 22 carbon atoms, G stands for a sugar group having 5 or 6 carbon atoms, and p stands for numbers from 1 to 10. They can be obtained according to relevant methods of preparative organic chemistry.

Alkylene oxide addition products to saturated, linear fatty alcohols and fatty acids, each with 2 to 100 mol of ethylene oxide per mole of fatty alcohol or fatty acid, have proven to be very especially preferred nonionic surfactants. Preparations with excellent properties are also obtained if they contain C₁₂-C₃₀ fatty acid monoesters and diesters of addition products of 1 to 30 mol of ethylene oxide to glycerol and/or addition products of 5 to 60 mol of ethylene oxide to castor oil and hydrogenated castor oil as the nonionic surfactants.

Very especially preferably, agents according to the invention contain as a surfactant at least one addition product of 15 to 100 mol of ethylene oxide, especially 15 to 50 mol of ethylene oxide, to a linear or branched (especially linear) fatty alcohol having 8 to 22 carbon atoms. This refers very especially preferably to ceteareth-15, ceteareth-25, or ceteareth-50, marketed as Eumulgin® CS 15 (COGNIS), Cremophor A25 (BASF SE), or Eumulgin® CS 50 (COGNIS).

All anionic surface-active substances suitable for use on the human body are, in principle, appropriate as anionic surfactants. These are characterized by an anionic group imparting water solubility, such as, e.g., a carboxylate, sulfate, sulfonate, or phosphate group, and a lipophilic alkyl group having approximately 8 to 30 C atoms. In addition, glycol ether or polyglycol ether groups, ester, ether, and amide groups, and hydroxyl groups can be contained in the molecule. Examples of suitable anionic surfactants are, each in the foul′ of the sodium, potassium, and ammonium and mono-, di-, and trialkanolammonium salts having 2 to 4 C atoms in the alkanol group,
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or is 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group, and sulfosuccinic acid monoalkylpolyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or is 1 to 12,
mixtures of surface-active hydroxysulfonates,
sulfated hydroxyalkylpolyethylene and/or hydroxyalkylenepropylene glycol ethers,
sulfonates of unsaturated fatty acids having 8 to 24 C atoms and 1 to 6 double bonds,
esters of tartaric acid and citric acid with alcohols, representing addition products of approximately 2 to 15 molecules of ethylene oxide and/or propylene oxide to fatty alcohols having 8 to 22 C atoms,
sulfated fatty acid alkylene glycol esters of formula (E1-II)

R⁷CO(AlkO)ₙSO₃M (E1-II)
in which R⁷CO stands for a linear or branched, aliphatic, saturated and/or unsaturated acyl group having 6 to 22 C atoms, Alk stands for CH₂CH₂, CHCH₃CH₂, and/or CH₂CHCH₃, n for numbers stands from 0.5 to 5, and M stands for a cation, as they are described in DE-OS 197 36 906,
amide ether carboxylic acids,
condensation products of C₈-C₃₀ fatty alcohols with protein hydrolysates and/or amino acids and derivatives thereof, known to one skilled in the art as protein fatty acid condensates, such as, for example, Lamepon® types, Gluadin® types, Hostapon® KCG, or Amisoft® types,
dimethicone copolyol.

Dimethicone copolyols according to the invention are understood to be preferably polyoxyalkylene-modified dimethylpolysiloxanes of general formulas (Si-1) or (Si-2):

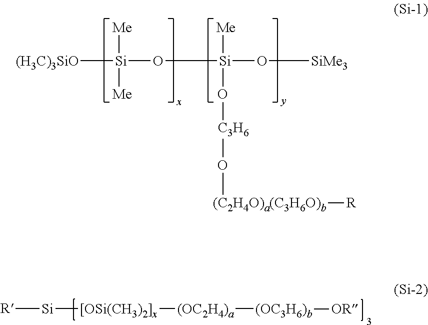

where
the group R stands for a hydrogen atom, an alkyl group having 1 to 12 C atoms, an alkoxy group having 1 to 12 C atoms, or a hydroxyl group,
the groups R′ and R″ denote alkyl groups having 1 to 12 C atoms,
x stands for an integer from 1 to 100, preferably from 20 to 30,
y stands for an integer from 1 to 20, preferably from 2 to 10, and
a and b stand for integers from 0 to 50, preferably from 10 to 30.

Especially preferred dimethicone copolyols within the meaning of the invention are, for example, the products commercially marketed under the trade name SILWET (Union Carbide Corporation) and DOW CORNING (Dow).

According to the invention, especially preferred dimethicone copolyols are Dow Coming 190 (INCI name: PEG/PPG-18/18 Dimethicone) and Dow Corning 193 (INCI name: PEG-12 Dimethicone) (Dow).

At least one cationic surfactant of the type of quaternary ammonium compounds, esterquat, amidoamines, or mixtures of these compounds are usable in addition according to the invention in the agents according to the invention. Nevertheless, the amounts are to be selected so that the stable incorporation of copolymers A and B into the cosmetic agent is preserved. Preferred quaternary ammonium compounds are ammonium halides, particularly chlorides and bromides, such as alkyltrimethylammonium chlorides, dialkyldimethylammonium chlorides, and trialkylmethylammonium chlorides. The long alkyl chains of these surfactants preferably have 10 to 18 carbon atoms, such as, e.g., cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Further preferred cationic surfactants are the imidazolium compounds known by the INCI names Quaternium-27 and Quatemium-83.

Surface-active compounds that have at least one quaternary ammonium group and at least one —COO⁽⁻⁾ or —SO3⁽⁻⁾ group in the molecule are called zwitterionic surfactants. Especially suitable zwitterionic surfactants are the so-called betaines, such as the N-alkyl-N,N-dimethylammonium glycinates, for example, coco alkyl dimethylammonium glycinate and N-acyl-aminopropyl-N,N-dimethylammonium glycinates, for example, coco acylaminopropyldimethylammonium glycinate, and 2-alkyl-3-carboxymethyl-3-hydroxyethyl imidazolines each having 8 to 18 C atoms in the alkyl or acyl group, and coco acylaminoethyl hydroxyethyl carboxymethyl glycinate. A preferred zwitterionic surfactant is the fatty acid amide derivative known under the INCI name of Cocamidopropyl Betaine.

Ampholytes are understood to be those surface-active compounds that contain at least one free amino group and at least one —COOH or —SO₃H group in the molecule, in addition to a C₈-C₂₄ alkyl or acyl group, and are capable of forming internal salts. Examples of suitable ampholytes are N-alkyl glycines, N-alkylpropionic acids, N-alkylaminobutyric acids, N-alkyliminodipropionic acids, N-hydroxyethyl-N-alkylamidopropyl glycines, N-alkyltaurines, N-alkylsarcosines, 2-alkylaminopropionic acids, and alkylaminoacetic acids each having approximately 8 to 24 C atoms in the alkyl group. Especially preferred ampholytes are N-cocoalkylaminopropionate, cocoacylaminoethyl-aminopropionate, and C₁₂-C₁₈ acylsarcosine.

The agents according to the invention absolutely comprise a cosmetically acceptable carrier. Preferred cosmetically acceptable carriers are aqueous, alcoholic, or aqueous/alcoholic media. Preferred cosmetic agents contain, based on their total weight, a water content of 30 to 97% by weight, especially preferably 40 to 95% by weight, very especially preferably of 50 to 90% by weight, and especially 60 to 90% by weight.

In particular, lower alcohols, usually used for cosmetic purposes and having 1 to 4 carbon atoms, such as, for example, ethanol and isopropanol, can be used as alcohols. Other organic solvents or a mixture of solvents can be contained as additional co-solvents. Unbranched or branched hydrocarbons such as hexane, isopentane, and cyclic hydrocarbons such as cyclopentane and cyclohexane are especially suitable as additional co-solvents.

The cosmetic agents preferably have a pH (25° C.) of 6.0 to 9.0, preferably 6.5 to 8.0, especially preferably 6.5 to 7.5. Agents with these preferred pH values have an especially good skin compatibility. Appropriate formulations because of their pH value are also suitable for packaging in metal aerosol containers.

In addition to the above-described copolymers and carrier substances, the cosmetic agents according to the invention can contain further ingredients. The group of these further ingredients include in particular the cosmetically effective adjuvants and additives.

Additional care substances can be named in particular as suitable adjuvants and additives.

For example, a silicone oil and/or a silicone gum can be used as a care substance.

Silicone oils or silicone gums suitable according to the invention are, in particular, dialkyl- and alkylarylsiloxanes, such as, for example, dimethylpolysiloxane and methylphenylsiloxane, as well as alkoxylated, quaternized, or also anionic derivatives thereof. Cyclic and linear polydialkylsiloxanes, alkoxylated and/or aminated derivatives thereof, dihydroxypolydimethylsiloxanes, and polyphenylalkylsiloxanes are preferred.

The agent can contain as a care substance of a different compound class, for example, at least one protein hydrolysate and/or a derivative thereof. Protein hydrolysates are product mixtures obtained by acid-, base-, or enzyme-catalyzed degradation of proteins. The term protein hydrolysates according to the invention is also understood to be total hydrolysates, as well as individual amino acids and derivatives thereof, and mixtures of different amino acids. The molecular weight of protein hydrolysates usable according to the invention is between 75, the molecular weight of glycine, and 200,000; the molecular weight is preferably 75 to 50,000 daltons, and very especially preferred 75 to 20,000 daltons.

The agent according to the invention can contain further at least one vitamin, provitamin, vitamin precursor, and/or a derivative thereof as a care substance. Those vitamins, provitamins, and vitamin precursors usually assigned to groups A, B, C, E, F, and H are preferred according to the invention.

Like the addition of glycerol and/or propylene glycol, the addition of panthenol increases the flexibility of the polymer film formed during use of the agent according to the invention.

The agents according to the invention, furthermore, can contain at least one plant extract but also mono- or oligosaccharides and/or lipids as a care substance.

Furthermore, oil components are suitable as care substance. Natural and synthetic cosmetic oil components include, for example, plant oils, liquid paraffin oils, isoparaffin oils, and synthetic hydrocarbons, as well as di-n-alkyl ethers with a total of 12 to 36 C atoms, especially 12 to 24 C atoms.

Further preferred nourishing oil components are ester oils, i.e., esters of C₆-C₃₀ fatty acids with C₂-C₃₀ fatty alcohols, preferably monoesters of fatty acids with alcohols having 2 to 24 C atoms, such as, for example, isopropyl myristate (Rilanit® IPM), isononanoic acid C₁₆-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V).

A preferred cosmetic agent according to the invention is characterized in that the cosmetic agent, based on its total weight, includes 0.01 to 5.0% by weight, preferably 0.02 to 4.0% by weight, and especially 0.05 to 2.0% by weight of an oil component.

Suitable as care substances, furthermore, are dicarboxylic acid esters, symmetric, asymmetric, or cyclic esters of carbonic acid with fatty alcohols, fatty acid triesters of saturated and/or unsaturated, linear and/or branched fatty acids with glycerol, or fatty acid partial glycerides, which are understood to be monoglycerides, diglycerides, and technical mixtures thereof.

The formulation of the agents according to the invention is applied to the hair very especially preferably as an aerosol foam. As a result, the agent according to the invention can include at least one propellant.

The agents according to the invention are packaged in a delivery device, which represents a pressurized-gas container (“aerosol container”) filled in addition with a propellant. The pressurized-gas container, with whose aid a product is dispensed by the inner gas pressure of the container via a valve, is called an “aerosol container” according to the definition. Conversely to the aerosol definition, a container under standard pressure with whose aid a product is dispensed by mechanical action using a pumping or squeezing system is defined as “non-aerosol container,”

The agents according to the invention can be produced in the usual manner. All components of the agent according to the invention, with the exception of the propellant, are usually introduced into a suitable pressure-tight container. The latter is then sealed with a valve. Lastly, the desired amount of propellant is introduced using conventional techniques.

The propellant is contained in the agents according to the invention preferably in amounts of 1 to 35% by weight, based on the total product. Amounts of 2 to 30% by weight, especially of 3 to 15% by weight, are especially preferred.

For a given spray device, the size of the foam bubbles and the respective size distribution can be adjusted using the quantitative ratio between the propellant and other ingredients of the preparations.

In the embodiment as an aerosol foam, propellants suitable according to the invention are selected, for example, from N20, dimethyl ether, CO₂, air, alkanes having 3 to 5 carbon atoms such as propane, n-butane, isobutane, n-pentane, and isopentane, and mixtures thereof. According to the embodiment of an aerosol foam, the above alkanes, mixtures of the above alkanes, or mixtures of the above alkanes with dimethyl ether are used preferably as the only propellant. The invention also expressly comprises, however, the concurrent use of propellants of the chlorofluorocarbon type, but particularly fluorocarbons.

Especially preferably, dimethyl ether, propane, n-butane, isobutane, or mixtures of at least two of these compounds form the propellant according to the invention. Very especially preferably, mixtures of propane and butane are used as the only propellant in the weight ratio of propane to butane of 70 to 30 to 15 to 85. These mixtures are in turn preferably used in the agents according to the invention in an amount of 3 to 15% by weight, based on the weight of the total agent. Butane according to the invention is understood to be n-butane, isobutane, and mixtures of n-butane and isobutane.

Isopentane is preferably suitable as a propellant for foaming of gel-type agents in a two-chamber aerosol container, with the propellant incorporated into the agents according to the invention and packaged in the first chamber of the two-chamber aerosol container. In the second chamber of the two-chamber aerosol container, there is at least one further propellant that is different from isopentane and in the two-chamber aerosol container builds up a higher pressure than isopentane. Propellants of the second chamber are preferably selected from N20, dimethyl ether, CO₂, air, alkanes having 3 or 4 carbon atoms (such as propane, n-butane, and isobutane), and mixtures thereof

Embodiments of the invention, preferred according to the invention, are particularly the embodiments (A) to (Z):

(A): Cosmetic agent, containing in a cosmetically acceptable carrier

a) a polymer combination of at least one copolymer A, comprising
(A1) at least one repeating structural unit with at least one silicone-containing side chain,
(A2) at least one structural unit of formula (a2)

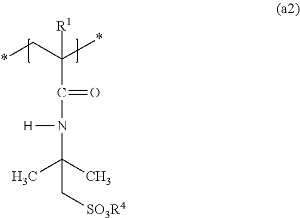

where R³ stands for a hydrogen atom or a methyl group and R⁴ stands for a hydrogen atom or an equivalent of a cation,
(A3) at least one structural unit of formula (a3)

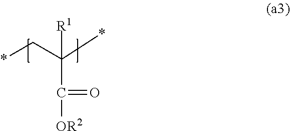

where R¹ stands for a hydrogen atom or a methyl group and
R² stands for a hydrogen atom, an equivalent of a cation, or a (C₁-C₄) alkyl group, and
at least one copolymer B of the monomers
   B1) n-butyl methacrylate
   B2) methacrylic acid
   B3) ethyl acrylate
   and optionally additional monomers, and
      b) at least one propellant,
      c) water,
      d) at least one (C₂-C₆) alkanolamine.

(B): Cosmetic agent, containing in a cosmetically acceptable carrier

a) a polymer combination of at least one copolymer A, comprising
(A1) at least one repeating structural unit with at least one silicone-containing side chain,
(A2) at least one structural unit of formula (a2)

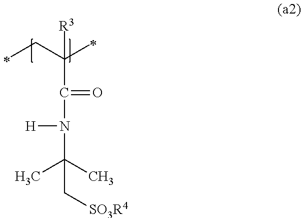

where R³ stands for a hydrogen atom or a methyl group and R⁴ stands for a hydrogen atom or an equivalent of a cation,
(A3) at least one structural unit of formula (a3)

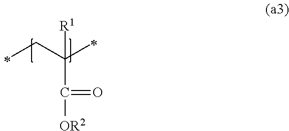

where R¹ stands for a hydrogen atom or a methyl group and
R² stands for a hydrogen atom, an equivalent of a cation, or a (C₁-C₄) alkyl group, and
at least one copolymer B of the monomers
   B1) n-butyl methacrylate
   B2) methacrylic acid
   B3) ethyl acrylate
   and optionally additional monomers, and
      b) at least one propellant,
      c) water,
      d) at least one (C₂-C₆) alkanolamine,
      e) at least one nonionic surfactant.

(C): Cosmetic agent, in a cosmetically acceptable carrier

a) a polymer combination of at least one copolymer A, comprising
(A1) at least one repeating structural unit with at least one silicone-containing side chain,
(A2) at least one structural unit of formula (a2)

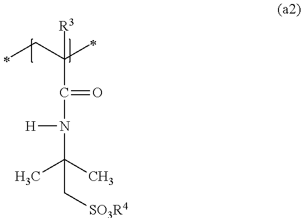

where R³ stands for a hydrogen atom or a methyl group and R⁴ stands for a hydrogen atom or an equivalent of a cation,
(A3) at least one structural unit of formula (a3)

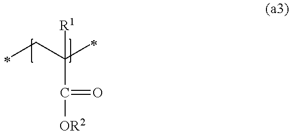

where R¹ stands for a hydrogen atom or a methyl group and
R² stands for a hydrogen atom, an equivalent of a cation, or a (C₁-C₄) alkyl group, and
at least one copolymer B of the monomers
   B1) n-butyl methacrylate
   B2) methacrylic acid
   B3) ethyl acrylate
   and optionally additional monomers, and
      b) at least one propellant, and
      c) water, and
      d) at least one (C₂-C₆) alkanolamine, and
      e) at least one nonionic surfactant, and
      f) at least one nonionic fixing polymer.

(D): Cosmetic agent, with a pH (25° C.) of 6.0 to 9.0, preferably of 6.5 to 8.0, especially preferably of 6.5 to 7.5, in a cosmetically acceptable carrier

a) a polymer combination of at least one copolymer A, comprising
(A1) at least one repeating structural unit with at least one silicone-containing side chain,
(A2) at least one structural unit of formula (a2)

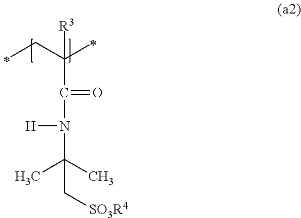

where R³ stands for a hydrogen atom or a methyl group and R⁴ stands for a hydrogen atom or an equivalent of a cation,
(A3) at least one structural unit of formula (a3)

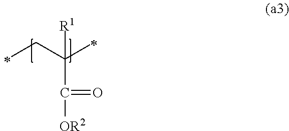

where R¹ stands for a hydrogen atom or a methyl group and
R² stands for a hydrogen atom, an equivalent of a cation, or a (C₁-C₄) alkyl group, and at least one copolymer B of the monomers
   B1) n-butyl methacrylate
   B2) methacrylic acid
   B3) ethyl acrylate
   and optionally additional monomers, and
      b) at least one propellant, and
      c) water, and
      d) at least one (C₂-C₆) alkanolamine.

(E): Cosmetic agent, with a pH (25° C.) of 6.0 to 9.0, preferably of 6.5 to 8.0, especially preferably of 6.5 to 7.5, in a cosmetically acceptable carrier

a) a polymer combination of at least one copolymer A, comprising
(A1) at least one repeating structural unit with at least one silicone-containing side chain,
(A2) at least one structural unit of formula (a2)

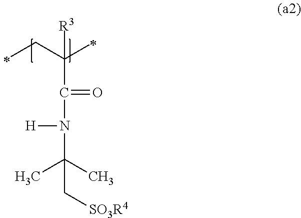

where R³ stands for a hydrogen atom or a methyl group and R⁴ stands for a hydrogen atom or an equivalent of a cation,
(A3) at least one structural unit of formula (a3)

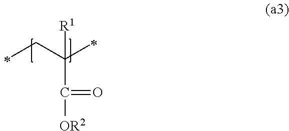

where R¹ stands for a hydrogen atom or a methyl group and
R² stands for a hydrogen atom, an equivalent of a cation, or a (C₁-C₄) alkyl group, and
at least one copolymer B of the monomers
   B1) n-butyl methacrylate
   B2) methacrylic acid
   B3) ethyl acrylate
   and optionally additional monomers, and
      b) at least one propellant, and
      c) water, and
      d) at least one (C₂-C₆) alkanolamine, and
      e) at least one nonionic surfactant.

(F): Cosmetic agent, with a pH (25° C.) of 6.0 to 9.0, preferably of 6.5 to 8.0, especially preferably of 6.5 to 7.5, in a cosmetically acceptable carrier

a) a polymer combination of at least one copolymer A, comprising
(A1) at least one repeating structural unit with at least one silicone-containing side chain,
(A2) at least one structural unit of formula (a2)

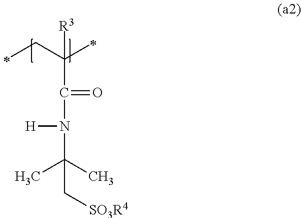

where R³ stands for a hydrogen atom or a methyl group and R⁴ stands for a hydrogen atom or an equivalent of a cation,
(A3) at least one structural unit of formula (a3)

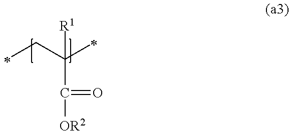

where R¹ stands for a hydrogen atom or a methyl group and
R² stands for a hydrogen atom, an equivalent of a cation, or a (C₁-C₄) alkyl group, and
at least one copolymer B of the monomers
   B1) n-butyl methacrylate
   B2) methacrylic acid
   B3) ethyl acrylate
   and optionally additional monomers, and
      b) at least one propellant, and
      c) water, and
      d) at least one (C₂-C₆) alkanolamine, and
      e) at least one nonionic surfactant.
      f) at least one nonionic fixing polymer.

(G): Cosmetic agent, in a cosmetically acceptable carrier
a) a polymer combination of at least one copolymer A, which is obtained from monomers (i), (ii), and (iii)
   (i) at least one monomer, carrying an olefinically unsaturated group and a silicone moiety, and
   (ii) at least one monomer of formula (a2-m),

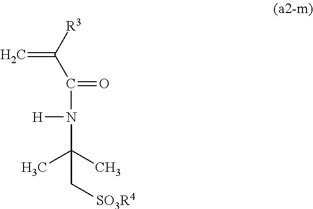

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers
   B1) n-butyl methacrylate
   B2) methacrylic acid
   B3) ethyl acrylate
   and optionally additional monomers, and
b) at least one propellant, and
c) water.

(H): Cosmetic agent, in a cosmetically acceptable carrier
a) a polymer combination of at least one copolymer A, which is obtained from monomers (i), (ii), and (iii)
   (i) at least one monomer, carrying an olefinically unsaturated group and a silicone moiety, and
   (ii) at least one monomer of formula (a2-m),

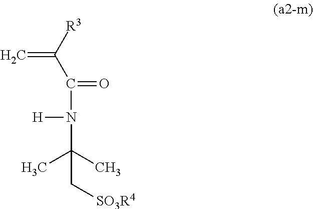

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers
   B1) n-butyl methacrylate
   B2) methacrylic acid
   B3) ethyl acrylate
   and optionally additional monomers, and
      b) at least one propellant, selected from dimethyl ether, propane, n-butane, isobutane, or mixtures of at least two of these compounds, and
      c) based on its total weight, a water content of 30 to 97% by weight, especially preferably 40 to 95% by weight, very especially preferably of 50 to 90% by weight, and especially 60 to 90% by weight, and
      d) at least one (C₂-C₆) alkanolamine, especially 2-amino-2-methylpropan-1-ol.

(I): Cosmetic agent, in a cosmetically acceptable carrier
a) a polymer combination of at least one copolymer A, which is obtained from monomers (i), (ii), and (iii)
   (i) at least one monomer, carrying an olefinically unsaturated group and a silicone moiety, and
   (ii) at least one monomer of formula (a2-m),

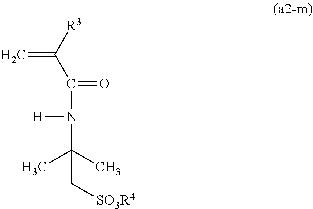

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers
   B1) n-butyl methacrylate
   B2) methacrylic acid
   B3) ethyl acrylate
   and optionally additional monomers, and
b) at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) at least one (C₂-C₆) alkanolamine, especially 2-amino-2-methylpropan-1-ol, and
e) at least one nonionic surfactant.

(J): Cosmetic agent, in a cosmetically acceptable carrier
a) a polymer combination of at least one copolymer A, which is obtained from monomers (i), (ii), and (iii)
(i) at least one monomer, carrying an olefinically unsaturated group and a silicone moiety, and
(ii) at least one monomer of formula (a2-m),

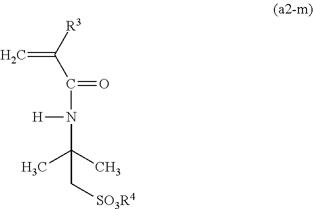

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers
   B1) n-butyl methacrylate
   B2) methacrylic acid
   B3) ethyl acrylate
   and optionally additional monomers, and
b) at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) at least one (C₂-C₆) alkanolamine, especially 2-amino-2-methylpropan-1-ol, and e) at least one nonionic surfactant, and
f) at least one nonionic fixing polymer.

(K): Cosmetic agent, with a pH (25° C.) of 6.0 to 9.0, preferably of 6.5 to 8.0, especially preferably of 6.5 to 7.5, in a cosmetically acceptable carrier, including
a) a polymer combination of at least one copolymer A, which is obtained from monomers (i), (ii), and (iii)
   (i) at least one monomer, carrying an olefinically unsaturated group and a silicone moiety, and
   (ii) at least one monomer of formula (a2-m),

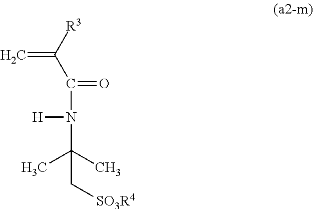

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers
   B1) n-butyl methacrylate
   B2) methacrylic acid
   B3) ethyl acrylate
   and optionally additional monomers, and
b) at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) at least one (C₂-C₆) alkanolamine, especially 2-amino-2-methylpropan-1-ol.

(L): Cosmetic agent, with a pH (25° C.) of 6.0 to 9.0, preferably of 6.5 to 8.0, especially preferably of 6.5 to 7.5, in a cosmetically acceptable carrier, including
a) a polymer combination of at least one copolymer A, which is obtained from monomers (i), (ii), and (iii)
   (i) at least one monomer, carrying an olefinically unsaturated group and a silicone moiety, and
   (ii) at least one monomer of formula (a2-m),

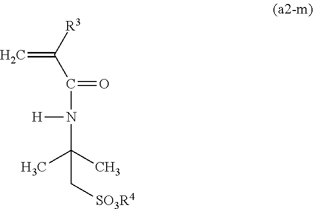

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers
   B1) n-butyl methacrylate
   B2) methacrylic acid
   B3) ethyl acrylate
   and optionally additional monomers, and
b) at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) at least one (C₂-C₆) alkanolamine, especially 2-amino-2-methylpropan-1-ol, and
e) at least one nonionic surfactant.

(M): Cosmetic agent, with a pH (25° C.) of 6.0 to 9.0, preferably of 6.5 to 8.0, especially preferably of 6.5 to 7.5, in a cosmetically acceptable carrier, including
a) a polymer combination of at least one copolymer A, which is obtained from monomers (i), (ii), and (iii)
   (i) at least one monomer, carrying an olefinically unsaturated group and a silicone moiety, and
   (ii) at least one monomer of formula (a2-m),

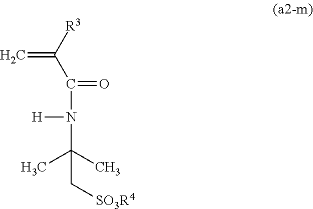

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers
   B1) n-butyl methacrylate
   B2) methacrylic acid
   B3) ethyl acrylate
   and optionally additional monomers, and
b) at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) at least one (C₂-C₆) alkanolamine, especially 2-amino-2-methylpropan-1-ol, and
e) at least one nonionic surfactant, and
f) at least one nonionic fixing polymer.

(N): Cosmetic agent, in a cosmetically acceptable carrier, including, based on its total weight, in a total amount of
a) 0.5 to 10% by weight, preferably from 3.0 to 8.0% by weight, of a polymer combination of at least one copolymer A, comprising
   (a1) at least one repeating structural unit with at least one a silicone-containing side chain,
   (a2) at least one structural unit of formula (a2)

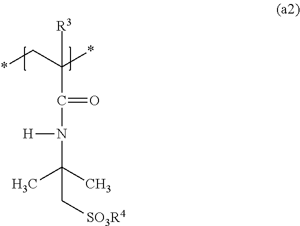

where R³ stands for a hydrogen atom or a methyl group and R⁴ stands for a hydrogen atom or an equivalent of a cation,
(a3) at least one structural unit of formula (a3)

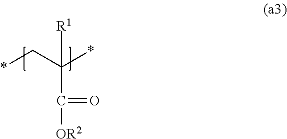

where R¹ stands for a hydrogen atom or a methyl group and
R² stands for a hydrogen atom, an equivalent of a cation, or a (C₁-C₄) alkyl group, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, and
c) water, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol.

(O): Cosmetic agent, containing in a cosmetically acceptable carrier, based on its total weight, in a total amount of
a) 0.5 to 10% by weight, preferably from 3.0 to 8.0% by weight, of a polymer combination of at least one copolymer A, comprising
   (a1) at least one repeating structural unit with at least one a silicone-containing side chain,
   (a2) at least one structural unit of formula (a2)

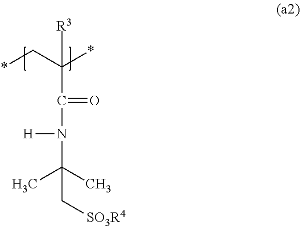

where R³ stands for a hydrogen atom or a methyl group and R⁴ stands for a hydrogen atom or an equivalent of a cation,
(a3) at least one structural unit of formula (a3)

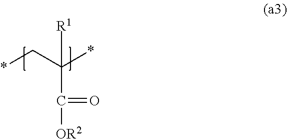

where R¹ stands for a hydrogen atom or a methyl group and
R² stands for a hydrogen atom, an equivalent of a cation, or a (C₁-C₄) alkyl group, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, and
c) water, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol, and
e) at least one nonionic surfactant.

(P): Cosmetic agent, including, in a cosmetically acceptable carrier, based on its total weight, in a total amount of
a) 0.5 to 10% by weight, preferably from 3.0 to 8.0% by weight, of a polymer combination of at least one copolymer A, comprising
   (a1) at least one repeating structural unit with at least one a silicone-containing side chain,
   (a2) at least one structural unit of formula (a2)

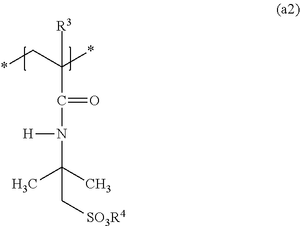

where R³ stands for a hydrogen atom or a methyl group and R⁴ stands for a hydrogen atom or an equivalent of a cation,
(a3) at least one structural unit of formula (a3)

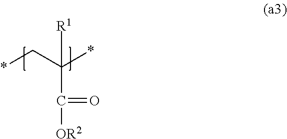

where R¹ stands for a hydrogen atom or a methyl group and
R² stands for a hydrogen atom, an equivalent of a cation, or a (C₁-C₄) alkyl group, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, and
c) water, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol, and
e) at least one nonionic surfactant, and
f) at least one nonionic fixing polymer.

(Q): Cosmetic agent, with a pH (25° C.) from 6.0 to 9.0, preferably from 6.5 to 8.0, especially preferably from 6.5 to 7.5, containing in a cosmetically acceptable carrier, based on its total weight, in a total amount of
a) 0.5 to 10% by weight, preferably from 3.0 to 8.0% by weight, of a polymer combination of at least one copolymer A, comprising
   (a1) at least one repeating structural unit with at least one a silicone-containing side chain,
   (a2) at least one structural unit of formula (a2)

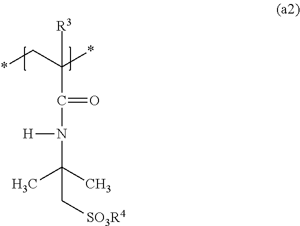

where R³ stands for a hydrogen atom or a methyl group and R⁴ stands for a hydrogen atom or an equivalent of a cation,
(a3) at least one structural unit of formula (a3)

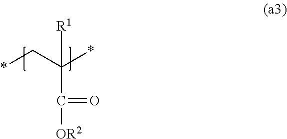

where R¹ stands for a hydrogen atom or a methyl group and
R² stands for a hydrogen atom, an equivalent of a cation, or a (C₁-C₄) alkyl group, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, and
c) water, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol.

(R): Cosmetic agent, with a pH (25° C.) from 6.0 to 9.0, preferably from 6.5 to 8.0, especially preferably from 6.5 to 7.5, containing in a cosmetically acceptable carrier, based on its total weight, in a total amount of
a) 0.5 to 10% by weight, preferably from 3.0 to 8.0% by weight, of a polymer combination of at least one copolymer A, comprising
   (a1) at least one repeating structural unit with at least one a silicone-containing side chain,
   (a2) at least one structural unit of formula (a2)

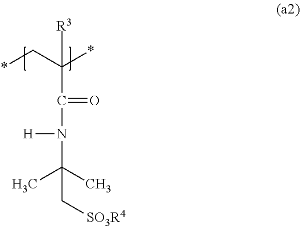

where R³ stands for a hydrogen atom or a methyl group and R⁴ stands for a hydrogen atom or an equivalent of a cation,
(a3) at least one structural unit of formula (a3)

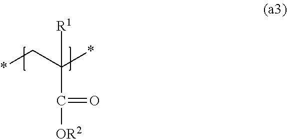

where R¹ stands for a hydrogen atom or a methyl group and
R² stands for a hydrogen atom, an equivalent of a cation, or a (C₁-C₄) alkyl group, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, and
c) water, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol, and
e) at least one nonionic surfactant.

(S): Cosmetic agent, with a pH (25° C.) from 6.0 to 9.0, preferably from 6.5 to 8.0, especially preferably from 6.5 to 7.5, containing in a cosmetically acceptable carrier, based on its total weight, in a total amount of
a) 0.5 to 10% by weight, preferably from 3.0 to 8.0% by weight, of a polymer combination of at least one copolymer A, comprising
   (a1) at least one repeating structural unit with at least one a silicone-containing side chain,
   (a2) at least one structural unit of formula (a2)

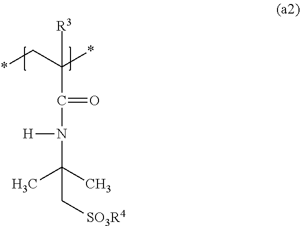

where R³ stands for a hydrogen atom or a methyl group and R⁴ stands for a hydrogen atom or an equivalent of a cation,
(a3) at least one structural unit of formula (a3)

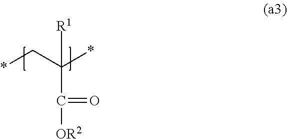

where R¹ stands for a hydrogen atom or a methyl group and
R² stands for a hydrogen atom, an equivalent of a cation, or a (C₁-C₄) alkyl group, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, and
c) water, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol, and
e) at least one nonionic surfactant, and
f) at least one nonionic fixing polymer.

(T): Cosmetic agent, containing in a cosmetically acceptable carrier, based on its total weight, in a total amount of
a) 0.5 to 10% by weight, preferably from 3.0 to 8.0% by weight, of a polymer combination of at least one copolymer A, which is obtained from the monomers (i), (ii), and (iii)
   (i) at least one monomer, carrying an olefinically unsaturated group and a silicone moiety, and
   (ii) at least one monomer of formula (a2-m),

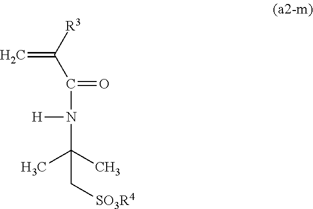

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, and
c) based on its total weight, a water content of 30 to 97% by weight, especially preferably 40 to 95% by weight, very especially preferably of 50 to 90% by weight, and especially 60 to 90% by weight.

(U): Cosmetic agent, containing in a cosmetically acceptable carrier, based on its total weight, in a total amount of
a) 0.5 to 10% by weight, preferably from 3.0 to 8.0% by weight, of a polymer combination of at least one copolymer A, which is obtained from the monomers (i), (ii), and (iii)
   (i) at least one monomer, carrying an olefinically unsaturated group and a silicone moiety, and
   (ii) at least one monomer of formula (a2-m),

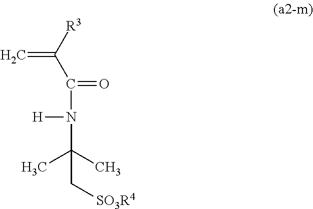

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
b) 1 to 35% by weight, preferably from 2 to 30% by weight, especially preferably from 3 to 15% by weight, of at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol.

(V): Cosmetic agent, containing in a cosmetically acceptable carrier, based on its total weight, in a total amount of
a) 0.5 to 10% by weight, preferably from 3.0 to 8.0% by weight, of a polymer combination of at least one copolymer A, which is obtained from the monomers (i), (ii), and (iii)
   (i) at least one monomer, carrying an olefinically unsaturated group and a silicone moiety, and
   (ii) at least one monomer of formula (a2-m),

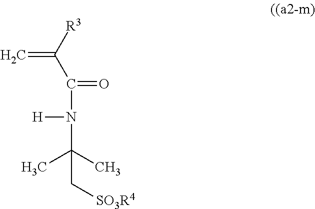

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol, and
e) at least one nonionic surfactant.

(W): Cosmetic agent, containing in a cosmetically acceptable carrier, based on its total weight, in a total amount of
a) 0.5 to 10% by weight, preferably from 3.0 to 8.0% by weight, of a polymer combination of at least one copolymer A, which is obtained from the monomers (i), (ii), and (iii)
   (i) at least one monomer, carrying an olefinically unsaturated group and a silicone moiety, and
   (ii) at least one monomer of formula (a2-m),

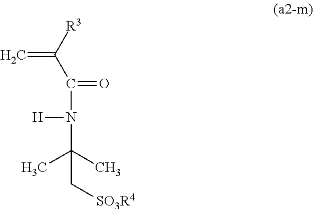

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol, and
e) at least one nonionic surfactant, and
f) at least one nonionic fixing polymer.

(X): Cosmetic agent, with a pH (25° C.) from 6.0 to 9.0, preferably from 6.5 to 8.0, especially preferably from 6.5 to 7.5, containing in a cosmetically acceptable carrier, based on its total weight, in a total amount of
a) 0.5 to 10% by weight, preferably from 3.0 to 8.0% by weight, of a polymer combination of at least one copolymer A, which is obtained from the monomers (i), (ii), and (iii)
   (i) at least one monomer, carrying an olefinically unsaturated group and a silicone moiety, and
   (ii) at least one monomer of formula (a2-m),

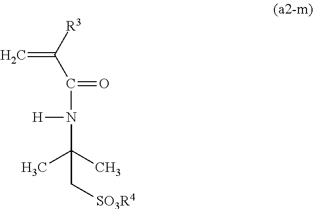

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol.

(Y): Cosmetic agent, with a pH (25° C.) from 6.0 to 9.0, preferably from 6.5 to 8.0, especially preferably from 6.5 to 7.5, containing in a cosmetically acceptable carrier, based on its total weight, in a total amount of
a) 0.5 to 10% by weight, preferably from 3.0 to 8.0% by weight, of a polymer combination of at least one copolymer A, which is obtained from the monomers (i), (ii), and (iii)
   (i) at least one monomer, carrying an olefinically unsaturated group and a silicone moiety, and
   (ii) at least one monomer of formula (a2-m),

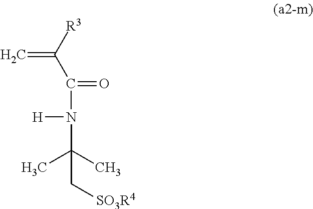

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol, and
e) at least one nonionic surfactant.

(Z): Cosmetic agent with a pH (25° C.) of 6.0 to 10.0. Especially preferably, the pH range is 7.0 to 9.0 and especially of 7.5 to 8.5, contained in a cosmetically acceptable carrier based on its total weight, in a total amount of
a) 0.5 to 10% by weight, preferably from 3.0 to 8.0% by weight, of a polymer combination of at least one copolymer A, which is obtained from the monomers (i), (ii), and (iii)
   (i) at least one monomer, carrying an olefinically unsaturated group and a silicone moiety, and
   (ii) at least one monomer of formula (a2-m),

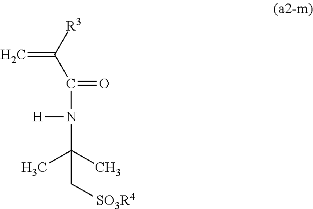

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt, and
at least one copolymer B of the monomers, based on the weight of the copolymer,
   a1) 60 to 80% by weight of n-butyl methacrylate
   a2) 10 to 30% by weight of methacrylic acid
   a3) 5 to 15% by weight of ethyl acrylate
   and optionally a maximum of 5% by weight of other monomers
b) 1 to 35% by weight, preferably from 2 to 30% by weight, especially preferably from 3 to 15% by weight, of at least one propellant, selected from dimethyl ether, propane, n-butane, isobutane, or mixtures of at least two of these compounds, and
c) based on its total weight, a water content of 30 to 97% by weight, especially preferably 40 to 95% by weight, very especially preferably of 50 to 90% by weight, and especially 60 to 90% by weight, and
d) 0.1 to 4.0% by weight, preferably 0.2 to 3.0% by weight, and especially 0.5 to 2.0% by weight of at least one (C₂-C₆) alkanolamine, especially 2-amino-2-methylpropan-1-ol, and
e) at least one nonionic surfactant, and
f) at least one nonionic fixing polymer.

The embodiments, cited as preferred above, of the features cited therein are again preferred mutatis mutandis for the preferred embodiments (A) to (Z). In particular, the polymer with the INCI name Polyacrylate-32 is especially preferred as copolymer A for embodiments (A) to (Z).

All agents of the first subject of the invention are to be foamed very especially preferably by delivery from an aerosol container. The resulting foam of the agent according to the invention is a very especially preferred application form.

A second subject of the invention is the use of a cosmetic agent of the first subject of the invention for increasing the volume of keratinous fibers, particularly human hair.

As set forth above, the agents according to the invention are notable particularly by an improved holding of the achieved volume of the keratinous fibers.

A third subject of the invention is a method for increasing the volume of keratinic fibers, particularly human hair, characterized in that a cosmetic agent of the first subject of the invention is applied in the form of a foam from an aerosol container to the keratinic fibers.

It is preferred according to the invention to apply the cosmetic agent of the first subject of the invention to the hairline area.

Further, it has proven preferable, if the keratinic fibers after exposure to the cosmetic agent of the first subject of the invention is not rinsed and the agent is left on the fibers.

A fourth subject of the invention is a product, comprising

i) a delivery device in the form of a pressurized gas container, and
ii) a cosmetic agent, within the delivery device, of the first subject of the invention.

A particular embodiment of the invention is described by the following points:
1. Cosmetic agent, containing in a cosmetically acceptable carrier
a) a polymer combination of at least one copolymer A, comprising
   (a1) at least one repeating structural unit with at least one a silicone-containing side chain,
   (a2) at least one structural unit of formula (a2)

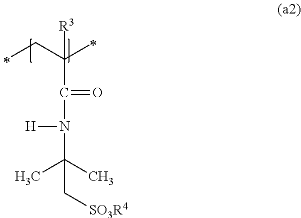

where R³ stands for a hydrogen atom or a methyl group and R⁴ stands for a hydrogen atom or an equivalent of a cation,
(a3) at least one structural unit of formula (a3)

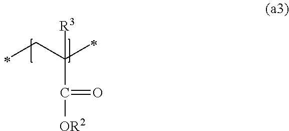

where R¹ stands for a hydrogen atom or a methyl group and
R² stands for a hydrogen atom, an equivalent of a cation, or a (C₁-C₄) alkyl group, and
at least one copolymer B of the monomers
   B1) n-butyl methacrylate
   B2) methacrylic acid
   B3) ethyl acrylate
   and optionally additional monomers, and
b) at least one propellant, and
c) water.
2. Cosmetic agent according to point 1, characterized in that the copolymer A is obtained from the monomers (i), (ii) and (iii)
   (i) at least one monomer, carrying an olefinically unsaturated group and a silicone moiety, and
   (ii) at least one monomer of formula (a2-m),

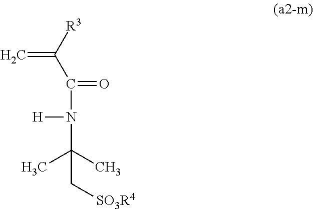

where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt.
3. Cosmetic agent according to point 1 or 2, characterized in that to preserve the structural unit (a1) preferably at least one monomer selected from the group, formed by compounds of formulas (A1) and (A2), is used as monomer (i)

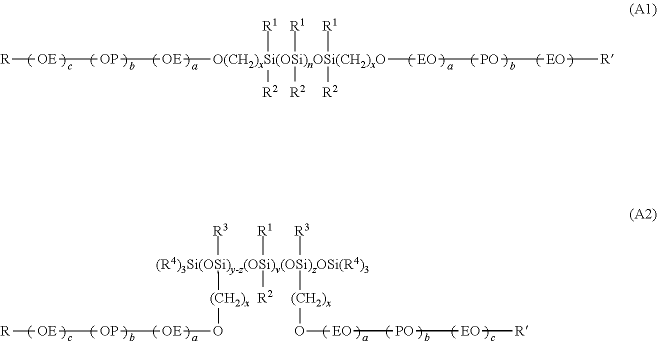

where
R¹ and R² independently of one another stand for a (C₁ to C₃₀) alkyl group, a (C₁ to C₂₀) alkyl group substituted with at least one halogen atom (e.g., —CCl3, —CBr₃, —CF₃), a (C₃ to C₈) cycloalkyl group, a (C₆ to C₁₄) aryl group, or (C₂ to C₂₀) alkenyl group,
R³ stands for a (C₁ to C₃₀) alkyl group, a (C₁ to C₂₀) alkyl group substituted with at least one halogen atom (e.g., —CCl₃, —CBr₃, —CF₃), a (C₃ to C₈) cycloalkyl group, a (C₆ to C₁₄) aryl group, or (C₂ to C₂₀) alkenyl group,
R⁴ independently of one another stands for a (C₁ to C₃₀) alkyl group, a (C₆ to C₁₄) aryl group, or a (C₂ to C₂₀) alkenyl group,
R and R′ at least one of the two groups R or R′ stands for an ester group of an olefinically unsaturated carboxylic acid according to one of formulas (Est1) to (Est7), whereby the other group also denotes one of these ester groups of formulas (Est1) to (Est7) or stands for a hydrogen atom,

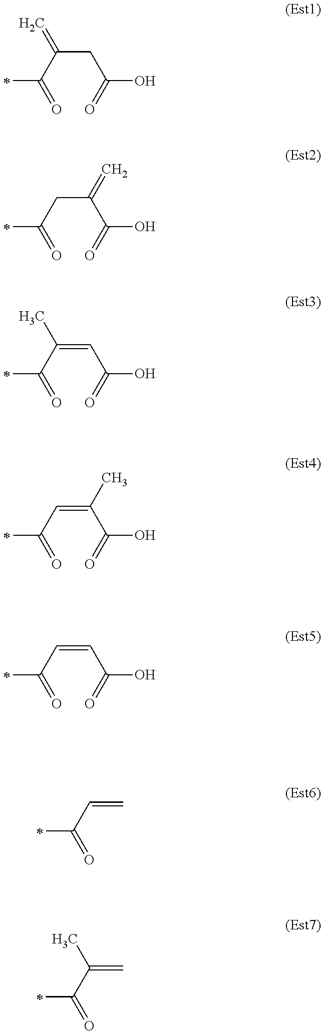

E stands for an ethane-1,2-diyl group,
P stands for a propane-1,2-diyl group or a propane-1,3-diyl group,
a, b and c independently of one another stand for a number from 0 to 100,
n stands for a number from 0 to 1000,
   x stands for a number 2 or 3,
   v stands for a number from 0 to 200,
   y stands for a number from 1 to 200, and
   z is less than or equal to y.
4. Cosmetic agent according to one of points 1 to 3, characterized in that copolymer A is polyacrylate-32.
5. Cosmetic agent according to one of points 1 to 4, characterized in that, based on its total weight, it contains copolymer A in an amount of 0.5 to 10% by weight, preferably of 3.0 to 8.0% by weight.
6. Cosmetic agent according to one of points 1 to 5, characterized in that copolymers B, based on the weight of copolymer B, contain 60 to 80% by weight of n-butyl methacrylate, 10 to 30% by weight of methacrylic acid, and 5 to 15% by weight of ethyl acrylate.
7. Cosmetic agent according to one of points 1 to 6, characterized in that, based on its total weight, it contains copolymer B in an amount of 0.5 to 10% by weight, preferably of 3.0 to 8.0% by weight.
8. Cosmetic agent according to one of points 1 to 7, characterized in that, based on the total weight of the cosmetic agent, the propellant is contained in amounts of 1 to 35% by weight, preferably of 2 to 30% by weight, especially preferably of 3 to 15% by weight.
9. Cosmetic agent according to one of points 1 to 8, characterized in that dimethyl ether, propane, n-butane, isobutane, or mixtures of at least two of these compound form the propellant according to the invention.
10. Cosmetic agent according to one of points 1 to 9, characterized in that, based on its total weight, it has a water content of 30 to 97% by weight, especially preferably 40 to 95% by weight, very especially preferably of 50 to 90% by weight, and particularly of 60 to 90% by weight.
11. Cosmetic agent according to one of points 1 to 10, characterized in that it has a pH (25° C.) of 6.0 to 9.0, preferably of 6.5 to 8.0, especially preferably of 6.5 to 7.5.
12. Cosmetic agent according to one of points 1 to 11, characterized in that the cosmetic agent contains at least one (C₂-C₆) alkanolamine (particularly 2-amino-2-methylpropanol).
13. Cosmetic agent according to point 12 characterized in that the total weight of the (C₂-C₆) alkanolamine (particularly 2-amino-2-methylpropanol) in the total weight of the cosmetic agent is 0.1 to 4.0% by weight, preferably 0.2 to 3.0% by weight, and especially 0.5 to 2.0% by weight.
14. Cosmetic agent according to one of points 1 to 13, characterized in that it contains in addition at least one fixing polymer different from copolymer A.
15. Cosmetic agent according to one of points 1 to 14, characterized in that it contains in addition at least one surfactant (especially at least one nonionic surfactant and/or at least one cationic surfactant).
16. Cosmetic agent according to one of the points 1 to 15, characterized in that, based on its total weight, it contains the polymer combination of copolymer A and copolymer B in a total amount of 0.5 to 10% by weight, preferably of 3.0 to 8.0% by weight.
17. Use of a cosmetic agent according to one of points 1 to 16 for increasing the volume of keratinous fibers, especially human hair.
18. Method for increasing the volume of keratinic fibers, particularly human hair, characterized in that a cosmetic agent according to one of points 1 to 16 is applied to the keratinic fibers in the form of a foam from an aerosol container.
19. Product comprising
   i) a delivery device in the form of a pressurized gas container, and
   ii) a cosmetic agent within the delivery device according to one of points 1 to 16.

### Examples

### 1.0 Effectiveness Tests

The following compositions were prepared by mixing the indicated raw materials (The quantitative data are percentage by weight, unless indicated otherwise):

| | E | V |
|---|---|---|
| Copolymer A * | 3.00 | — |
| Copolymer B ** | 2.50 | — |
| Cetyltrimethylammonium chloride | — | 1.00 |
| 2-Amino-2-methylpropanol | To pH 7.5 | To pH 7.5 |
| Propane/butane | 8.00 | 8.00 |
| Water | To 100 | To 100 |

* 32% by weight aqueous solution of the polymeric active substances (Fixate ® Design, INCI name: Polyacrylate-32)
** 40% by weight aqueous dispersion of a terpolymer of monomers a1) n-butyl methacrylate, a2) methacrylic acid, and a3) ethyl acrylate

These compositions can be tested by means of a ring test and the hairline volume measurement with respect to its hair volume.

### 1.0 Ring Test

Standardized hair strands from Kerling, hair type “European natural,” color 7/0 (Batch No.: 04/2012, B3), with a length (Lₘₐₓ) of 220 mm and a weight of 2.8 g were used. For preparation, the strands were dyed medium blonde with a commercial bleach (Poly Blonde). The hair strands were stored for at least 48 h at 25° C. and 25% relative humidity.

In each case, 0.15 g of compositions E and V per gram of hair was applied to a wet hair strand. Each untreated reference strand and the strands treated with the test compositions were fixed at both ends in the holder of a torsion device. The top strand end was twisted 5 times around the strand axis. The strand was blow dried dry for 45 minutes in this position. The strands in the twisted state were then stored for 16 h at 50% relative humidity and 23° C. and removed from the holder. The strands were then combed 5 times with the wide teeth and 5 times with the fine teeth of a comb (Hercules Sagemann, hard plastic comb with fine tine).

Five hair strands were measured per composition and the arithmetic mean of the measured value, determined as follows, was obtained: The combed strand was mounted by hanging immediately after combing in the measuring cell of the measuring device (stress-strain system MTT 175 with ring (diameter: 10 mm); control unit: UV 1000 (Dia-Stron Ltd, UK)). The top end of the strand is inserted in the ring and the strand is then pulled through the ring at a speed of 100 mm/min. In this case, the force necessary to accomplish this and the covered distance are recorded.

The greater the required force, the more voluminous the hair strand.

### 2.0 Hairline Volume Measurement

Standardized hair strands from Kerling, hair type “European natural,” color 7/0 (Batch No.: 2010), with a length (Lₘₐₓ) of 8 cm and a weight of 2.3 g were used.

For preparation, the strands were washed for 30 minutes with a 12% by weight aqueous solution of sodium lauryl ether sulfate (pH 6.5), rinsed with tap water for 5 minutes, and then combed. The hair strands were dried overnight at 21° C. and 50% relative humidity in the climatic chamber. This hair was introduced into a facsimile of a natural scalp, in which smaller hair fiber bundles are arranged in a row on a plastic support bent 90° in a circular fashion and fixed there with one end. The hairline is simulated in this way. The other end of the fiber hangs free.

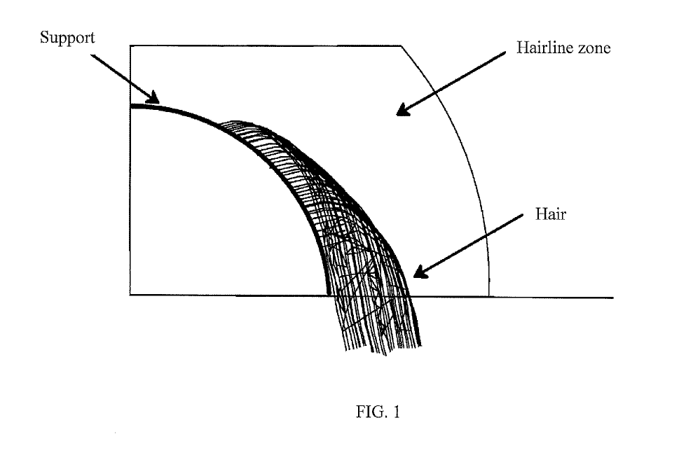

Before the application of the test composition, the plastic support was fixed, so that the plastic support described a 90° arc pointing downward (like the “12 to 3 o'clock” section of a wall clock, cf. FIG. 1), and the hair is located on the upward pointing surface of the support. The hair strand was photographed with a black/white CCD camera with a 50 mm lens. The camera was calibrated beforehand with a black disk.

The supported strand was moistened with water and a towel with a weight of 2 kg was pressed on for 30 seconds for drying. 10 mL (0.375 g) of compositions E and V were applied to a supported hair strand in each case and distributed in the hair with a brush. Next, the supported strands were combed 20 times with a comb with the wide teeth of a comb (Hercules Sägemann, hard plastic comb with fine tine).

Five hair strands were prepared per composition and simultaneously fixed as described above and dried for 7 minutes at 40° C. Next, the strands were brushed twice with a brush (this brush was provided with an ionizer against static charges) and dried for a further 7 minutes at 40° C. The supported strands were cooled for 5 minutes at room temperature and photographed as described above with a black/white CCD camera with a 50 mm lens.

The photographs were all taken as two-dimensional black and white images (background: black, hair and support: white). A morphological filter was used with a threshold of 210 in the hairline zone in order to fill black spots within the hair. The white area within the hairline zone of the filtered photographs of the supported strand were calculated and compared in each case before the treatment and after the treatment. The percentage of change in the area was calculated. The greater the change, the more voluminous the hair strand.

$$Change in \% = \frac{area ( treated strand ) - area ( untreated strand )}{area ( untreated strand )} \cdot 100$$

Five hair strands were measured per composition and the arithmetic mean was determined.

| | |
|---|---|
| Copolymer A * | 3.50 |
| Copolymer B ** | 3.50 |
| PVP/VA copolymer | 1.40 |
| Sodium benzoate | 0.30 |
| Water lily flower extract | 0.50 |
| Stinging nettle extract | 1.30 |
| Benzophenone-4 | 0.10 |
| D-Panthenol | 0.185 |
| Glycerol | 0.20 |
| Dow Corning 939 | 0.40 |
| Cocamidopropyl betaine | 0.50 |
| PEG -40 Hydrogenated castor oil | 0.80 |
| Perfume | 0.15 |
| Propane/butane | 8.00 |
| 2-Amino-2-methyrpropanol | To pH 7.5 |
| Water, demineralized | To 100 |

* 32% by weight aqueous solution of the polymeric active substances (Fixate ® Design, INCI name: Polyacrylate-32)
** 40% by weight aqueous dispersion of a terpolymer of monomers a1) n-butyl methacrylate, a2) methacrylic acid, and a3) ethyl acrylate

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent, in a cosmetically acceptable carrier, comprising:
a) a polymer combination of at least one copolymer A, comprising
(a1) at least one repeating structural unit with at least one a silicone-containing side chain,
(a2) at least one structural unit of formula (a2) 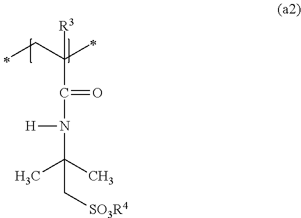
where R³ stands for a hydrogen atom or a methyl group and R⁴ stands for a hydrogen atom or an equivalent of a cation,
(a3) at least one structural unit of formula (a3) 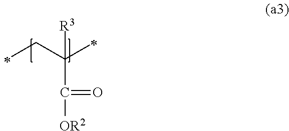
where R¹ stands for a hydrogen atom or a methyl group and
R² stands for a hydrogen atom, an equivalent of a cation, or a (C₁-C₄) alkyl group, and
at least one copolymer B of the monomers
B1) n-butyl methacrylate
B2) methacrylic acid
B3) ethyl acrylate
and optionally additional monomers, and
b) at least one propellant, and
c) water.

2. The cosmetic agent according to claim 1, wherein copolymer A is obtained from the monomers (i), (ii) and (iii)
(i) at least one monomer, carrying an olefinically unsaturated group and a silicone moiety, and
(ii) at least one monomer of formula (a2-m), 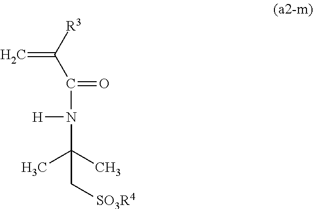
where R³ stands for a hydrogen atom or a methyl group and R⁴ stands for a hydrogen atom or an equivalent of a cation, and
(iii) at least one additional monomer selected from the group, formed by (C₁-C₄) alkyl acrylate, (C₁-C₄) alkyl methacrylate, acrylic acid, acrylic acid salt, methacrylic acid, and methacrylic acid salt.

3. The cosmetic agent according to claim 2, wherein the at least one monomer carrying an olefinically unsaturated group and silicone moiety (i) is formed by compounds of formulas (A1) and (A2), 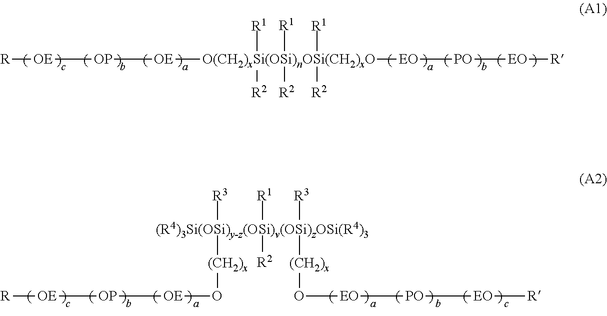
where
R¹ and R² independently of one another stand for a (C₁ to C₃₀) alkyl group, a (C₁ to C₂₀) alkyl group substituted with at least one halogen atom (e.g., —CCl₃, —CBr₃, —CF₃), a (C₃ to C₈) cycloalkyl group, a (C₆ to C₁₄) aryl group, or (C₂ to C₂₀) alkenyl group,
R³ stands for a (C₁ to C₃₀) alkyl group, a (C₁ to C₃₀) alkyl group substituted with at least one halogen atom (e.g., —CCl₃, —CBr₃, —CF₃), a (C₃ to C₈) cycloalkyl group, a (C₆ to C₁₄) aryl group, or (C₂ to C₃₀) alkenyl group,
R⁴ independently of one another stands for a (C₁ to C₃₀) alkyl group, a (C₆ to C₁₄) aryl group, or a (C₂ to C₂₀) alkenyl group,
R and R′ at least one of the two groups R or R′ stands for an ester group of an olefinically unsaturated carboxylic acid according to one of formulas (Est1) to (Est7), whereby the other group also denotes one of these ester groups of formulas (Est1) to (Est7) or stands for a hydrogen atom, 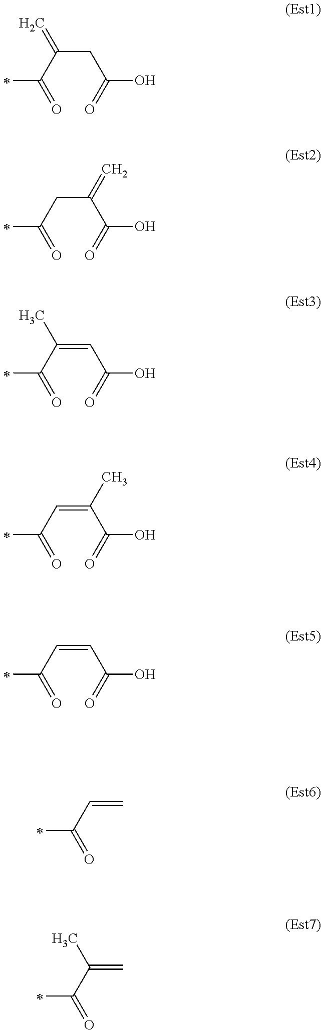
E stands for an ethane-1,2-diyl group,
P stands for a propane-1,2-diyl group or a propane-1,3-diyl group,
a, b and c independently of one another stand for a number from 0 to 100,
n stands for a number from 0 to 1000,
x stands for a number 2 or 3,
v stands for a number from 0 to 200,
y stands for a number from 1 to 200, and
z is less than or equal to y.

4. The cosmetic agent according to claim 1, wherein copolymer A is polyacrylate-32.

5. The cosmetic agent according to claim 1, wherein the agent includes 0.05% to 10% by weight of copolymer A.

6. The cosmetic agent according to claim 1, wherein the agent includes 3.0 to 8.0% by weight of copolymer A.

7. The cosmetic agent according to claim 1, wherein the copolymer B comprises 60 to 80% by weight of n-butyl methacrylate, 10 to 30% by weight of methacrylic acid, and 5 to 15% by weight of ethyl acrylate, based on the total weight of copolymer B.

8. The cosmetic agent according to claim 1, wherein the agent comprises 0.5% to 10% by weight of copolymer B, based on the total agent weight.

9. The cosmetic agent according to claim 1, wherein the agent comprises 3.0 to 8.0% by weight of copolymer B, based on the total agent weight.

10. The cosmetic agent according to claim 1, wherein the cosmetic agent comprises 30 to 97% by weight water, based on the total weight of the cosmetic agent.

11. The cosmetic agent according to claim 1, wherein the cosmetic agent comprises 40 to 95% by weight water, based on the total weight of the cosmetic agent.

12. The cosmetic agent according to claim 1, wherein the cosmetic agent comprises 50 to 90% by weight water, based on the total weight of the cosmetic agent.

13. The cosmetic agent according to claim 1, wherein the cosmetic agent comprises 60 to 90% by weight water, based on the total weight of the cosmetic agent.

14. The cosmetic agent according to claim 1, wherein the cosmetic agent has a pH at 25° C. of 6.0 to 9.0.

15. The cosmetic agent according to claim 1, wherein the cosmetic agent has a pH at 25° C. of 6.5 to 8.0.

16. The cosmetic agent according to claim 1, wherein the cosmetic agent has a pH at 25° C. of 6.5 to 7.5.

17. The cosmetic agent according to claim 1, wherein the sum of the weight percent of copolymer A and copolymer B is 0.5% to 10% by weight, based on the total weight of the cosmetic agent.

18. The cosmetic agent according to claim 1, wherein the sum of the weight percent of copolymer A and copolymer B 3% to 8% by weight, based on the total weight of the cosmetic agent is.
